SEQUENCE LISTING (124 sequences)

SEQ ID NO: 1 - DNA, 501 bp, Bos taurus
  misc_feature (251)..(251): n is either A or G
cagccagggtcctctgtccatgggattctccaggcaagactactggagtgggtagccattccattctcccggggatcttccctacccagggatcaaacccgagactcttatgtctcctgtactggcaggtgggttctttattactactgccattggagaagccccaaccttttcttcagagaagattgcaaataccaaaaacctatagtgaagtgagatatatacaagcctggggaagtaccaagaaagcngaatatgatagaaacaggaagaagttattaaaaggtgccagacctcaaacagaagcagactctttcagcatcagccaacttcctctctcctccccaaagctgcagattttacaaaattgaatttcagtgtctttgatagaaggattagagggacacatacctaaagacatatgtgttattactattactattttaaactactactgttatttactactacttctgttattattattatttttagatttgt

SEQ ID NO: 2 - DNA, 420 bp, Bos taurus
  misc_feature (240)..(240): n is either A or G
tgcccccaaatgagaacaaattattggcatataactttaagaatagcataaatgtgtacatttgaaatgaaacgaatgtgtcttgaatcctcatacattttcttaccagtcccgtctattttgtctttgatccaaactcctaaatgtttgtgcacatgttttgtggtgacaatgctgggaaacacagcaacaggacttcattattctgttccttcctgtcattatggaaaccagtcatcnacctatggcgtgatggcaggtaagaaaaattgtctttacatgtaagattgagtttggggacgcttggatgcattttctgggtcgaagggaatcttgaccagagtgtatcatgaaattcagatctcctaaccttagaaattgctgctaaatccaccacttactataatggtccctgatctg

SEQ ID NO: 3 - DNA, 501 bp, Bos taurus
  misc_feature (251)..(251): n is either C or T
aataatgaaaaccacagtagctattaagatatgcctgggtcaagattcccagaaaatgactctaatatacagatttttgaagtttactggaggttattctcaggaatactacttaaaagtgggagatgaaaccatgttgggatgaaggacaacttgaattatgttacaattgtgatatagaccactattgatcccatggggagttaacaagtcacatggtcttcaaacagtctccagttgatctaaagaanagttacagtttacccccatgtcaaccttattgaaaacaggcagttatttttggttgagggtaattcctcaagcagcatccagctatgatgtagacatacagtctattagatagaatagacttacagtctattagactagtagactaattttgtctattagtagacaaaatgaacaagctaatggggaaatcagtacctaggtcctgaagcagggagtttggagcagtatccactgcattagatacagtag

SEQ ID NO: 4 - DNA, 501 bp, Bos taurus
  misc_feature (251)..(251): n is either C or T
tattagatatcatgaatattcaaagtactttttgactacaggaaattactgaaaatcttgccacagtagtggtttcaatacataatcaaaataaaaatattggtctggttgctaaaatgtaatacatttgctctgatgtagctcctttgagtataatagaaaaatgtagattaaaaatactttttgtcatcttcaatttcatgttatcagatttcaaacaagaggcctgtcttcatattgcttttcagtanactctgaaatccaccttttttttttaaagtatattttttttagacagctgccagttgagttctaatttcacattccatttttttcctggcaacacctttgagaaacatatgcttccaacttcagctactgctttttgctccatatgcctcctgttccatttctaggtgtcaggaaggcacttgggctggttatgtgcatgattaagttcatggagcttcttggaggaatcaggccctccaagttggaggg

SEQ ID NO: 5 - DNA, 501 bp, Bos taurus
  misc_feature (251)..(251): n is either C or T
aaaaccattctatctagatatgggagaaggaaagacattttctgtatttagtgtgcctaattgtgttaaagacacacagaattcatattcttactcaaaatattccatacacaccacatctcaatctacagttaactgcatttgtgttatgtcagagtcagatcttgagtgcaaatttccaccagtttcattttgctatacacatgtgaaaataaaagacacattagaaatttatgctaaggactgttttncccaaatgtatgactttcaagtccaaggaatgaaaactgttgttttgttccaagtggttaagtctaagctacaccaagcaccccagtgtattaactaacacattcaggttcatttccaaaattaagatgtgttgatggaattcaccctacccatcagaaatatcttcacatgtaaatcaaaagttgttcgtggtatttgtatgtgtgtattatattcctctttataatccatcctctcagtgatggtgat

SEQ ID NO: 6 - DNA, 501 bp, Bos taurus
  misc_feature (251)..(251): n is either C or T
atcacaaatcaagttcaatgtaccaaagccctcattttcatgaagactggggccaaccctggacatgttcagtcactagcaaccattctcaaagtccctcgatgtaacccaggctctcctgactcctggtgcacctcagctatgatctccaggtatgtcatttctacataaagccacctgctccccccattgccacccccgcccctggcgcacattgtatcatggtgcactggaagatgccctagttctanacctagaaaaactgaacagaaaagcaagtctcttctgagctgtctcagaagtcatccaaggagagttcctccataacgcacagcaaaacattctgaacattctccttctcagctcctcacagacatggcaggcacttcctagttacttttctcaccctcctaactccccaattctcctctcagttgccccctatctctggctgtccctttttcttataactttcttataacttataactttcttataact

SEQ ID NO: 7 - DNA, 501 bp, Bos taurus
  misc_feature (251)..(251): n is either A or C
tacagtccatgtttaaataatcaactagatgatgaacccatgagcaagcctttatggtttcagatcttagttatttctattttaattctcaattcagcaaaagaatcacactgcagcttggtaatatgcattaattcatatgccctaaactgctgacagagacattgttatgtagcaaaagataacatgcattctattcatgtctgttttgagactgtgttaaattaaaatctttagaaagccaatccctntaatactaggtcttcctattcaagcacactgcacatttctcatttacttaagtattctgtgtgtctctcagtagcatttcaaagttttctttcacaagataatacattttttgatactactgagaatgaaatcttttcctttactatgttttgcaatgggttgtttacgtacagaaaggtattttatccatttgtgtttctaaattctcttacattttggtattggtttttgattgatcattttggattt

SEQ ID NO: 8 - DNA, 501 bp, Bos taurus
  misc_feature (251)..(251): n is either A or G
ggtctgggtatcaagttgtcctctgaggcataagaaccgagttatccacagccatctcttcacgtccagcggctgcccctatcaaccaaaagatgaccatctctagcatctccagttcctgtgctctcctggggttccgatctccacatatcataccacacgatgcccagaatggagcagaaattctttccatctattatggaatccttcccccatttccccaaaatagcagccttggtggcatctttatntggggttatcgggaaacccagatactgttttcttagaattatttattatggcctcgtaattagcctcccctttcagtctcgccccactccagacagtcttccattctgcttctagagttactttcctctcaatttagtctgattatatcactctcctgatgaaagatccctgactcctatttgtcttccctacagggcaggagccagttcctttcaagtgtctagcaactgggccttgaatgcagtagac

SEQ ID NO: 9 - DNA, 501 bp, Bos taurus
  misc_feature (251)..(251): n is either C or T
gctcctgttcctcttttcattgttacaataattatattgtgtgtacattcgaagtcacttcagttgtgtctgactctttgcaacctcacagatcatagcccaggaggctcctctgtccatggggttctccaggcaagaaaaatggagtgggttgccatgccctcctccaggggatcttccctatccagggatcgaacccatgtctcttagtctcctgcattggcaggagggttctttactactagtgccantgggaaactccataatctactaattcatcttcctcatttccatccctctctatacctagcagctacagtgattggtttacaaatgaaaatcagatcatatttttgcttgcctatttgaaacttttcagtggcttcccatctccctttggaaataaaaggttttttttttttttcaaagagtttatactttattctgcagaggataatgagctgtgaaagggtttaatccccaaagtgtcactgttaggta

SEQ ID NO: 10 - DNA, 501 bp, Bos taurus
  misc_feature (251)..(251): n is either A or G
aattgcataataaagggcaaggaggccttttctaaactttatattttgggaatctgtattgaagacctacaattagaaatgatagatttatagccaaagttaagattaccattggatcttagatctccctaagcgtaggaaagttctactcacaacccctgccccaatctcaaggaactcacattcggacataaaactgtatgactttctttcttaattctttgtccctttacagtttctaatgacgtcantgagtttgagctagctgattcatactgtctttatggttctttctattcctccccaaagcggaaaaatctaaacacaaataaaaggcatactggaaggtttatttttctgcaagagtttataataaatgaaactgactcagatgttgaaaattgtccatgactgtaaactttaaagaaatttcagaatccctatgaaaaataaaccttaatctcctaaaaagtactttgtagtttactttttcttagttta

SEQ ID NO: 11 - DNA, 101 bp, Bos taurus
  misc_feature (51)..(51): n is either C or T
ggcaaagagggtgacatagacctgtcataaaagatgtttcccatacaaacntaagtctccacttcagaacccagttgagaaagtcattcctcatcttattt

SEQ ID NO: 12 - DNA, 501 bp, Bos taurus
  misc_feature (251)..(251): n is either C or T
ggtgataccatacaaccatctcatcgtttgtcatctccttctcctcccaccttcagtctttcccagcatcagggtcttttccaatgagtctgttctttgcatcaggtggccaaacatcatccctaacacttgttatttgttgtctttttgatgatagctattctgaccggtatgaggtgatatctcactgttttgatttgcatttctctgacgattagtgatgttggcatctttttatgttggccaccttntttggaaaaatgtctattcaggtcctctgcctatttttaaatcaggttatttgttttttctttgatgttgagttgtatgagttctttgtatgctttcaatattaacccctatcagatagtgtttgcaaatatccttttgctttgttcatagtttcttttgctatgcaaaagatttttagttttatttagttccatttgcttatttttgctcatttctctttcctggttttcaaagccagacattctgagg

SEQ ID NO: 13 - DNA, 501 bp, Bos taurus
  misc_feature (251)..(251): n is either A or C
ttctgaattgattagaatctgtgtgcagacctcttaacttcttggcgccagtggttaaaggaaatgtgtgttaggaagtcccaggagaattgctaggcttgatgtacagtgccaggcataagtgttctaaagttttggtgtctgctgtctctagatcatcatgtaaaattacatgtttttatttcccaataaatttgtgatggtattaggtcaatatttctggattttaactgttacacatggactaagcnctagatgatcccaagtattgctttatgaagacagttctgctagcatttttaaaatttgtgttctctttaagtatctttataaaggatcattgctttataaatctttggaaatatattttagtttgtcaagttacttcagttctgcagtaaacaagttttgagggtaaattaagttttgaattttcattaaaataagattacctcatactcatgataaaagaatgatctcttaatatattattatggagtt

SEQ ID NO: 14 - DNA, 501 bp, Bos taurus
  misc_feature (251)..(251): n is either C or T
acatccagtgttgcacctttgcatccataacacactgtctgaggattttttttattaccctgtcgagacaaagagcaatgggctgcatttcaattcaagacccaacagactgcagtgctgagtctgaatccttcatggcagcctctgctctagctctgccagctgcccccaggttgtctatactgggtgtttccgttgccctccatccccaccttctctgtcttccatccagtgctgggcatcaggtgtanttagatgccagaagaaaagacggagatggtcaggtgtgggtccagactgccttagagctcaggaatgtctgagagtaaagagggctggagcaggtggggacatcttcctggaggtgatgggccttgataacaagatttagcaaggtcctgagctcacaatttacaaggctctcacatacagacagggtctcaatgaatcatcacaacaaccctgtgaaggaagaacaattattctcattttccagcttag

SEQ ID NO: 15 - DNA, 501 bp, Bos taurus
  misc_feature (251)..(251): n is either C or T
aaccactcacatgcaaaatgtatgtaaccactcacaaatcaagctgtattctgtgtgtgtcatactgtgaatatggattttctgggtcaagcaaagaatgtctgtgagatgaaggtttagaagtaagccacgtaactctgcgtcagcacaattctctgattgtatttttatcacctgaatacactcacctttcaaccctgaccatgatttatctctaagtctctcggtgcatggaaaataagctgtaaacncctttctgcctcatataatgtcctaatttttcctattccagtatttcctcattcttcttttccccacccacttttccagccccaaatcattatcctcattcttttacataaaattccctttcaacaatcctttccctctcatttccattcctctccttgtgaccgccatgacccaatcccctgagggtgtacttgtgcttaatcctgtctattgttcataacttctttctcctctgggttctctgcctcc

SEQ ID NO: 16 - DNA, 501 bp, Bos taurus
  misc_feature (251)..(251): n is either A or C
tgcaagaatactggagtgggttgcaatgccctgctccagggaatcttcccaacccagggatcgaaccacggtctcctgcattgcaggcagattctttaccattcgagctacctgggaagcccagccctgtactttaattccaatcaatgatttcctccaacagtttttatgggtaactccaagagaactccagagaagttgcagtgcaggcaaagagaaaccatttgtctctgggtacaaagcttctctgngcagctcatgctctgtactgctttttaaactcatgatagcaccaaggctgaaaaccaacacccttaagataaatatagcaccagattggagtgcctttagtagtcagcaaagataaatgtttaacagtgtattcaaaacaaagtgttttatgttaaatcaaatagaagctaaatactaataaagaactctacaaatgaacaataaagtaatcagaataagtactcacattagcaggatattgatgttatt

SEQ ID NO: 17 - DNA, 501 bp, Bos taurus
  misc_feature (251)..(251): n is either A or G
cactcagttttagcaaacaaaaatgccagtctccatggaagtttctcccccgttttctgtttcaaattttagttctttgattttagaaacgaatttagactactaaattcttaattatgtttgtacaacaaagaaccaagatttatcatatctcaaatttgatgttgaatgcctagtaatatagacttcaaataaacctcctcttggaacttctctattacagcatctcatgtgaagacttcaagatatcntattcacctccgtgaacattccttgtcaaaatacctgcctgtcacctcccttcacaagacaccactcagtagaccatcttgggctttacctgtttagagtcctgctctcctctctaacttcctcaaggacaaaatcaatgttcattcattgttctgttcccagtaactagcccagttcctgaaacattctgtgtaggtgcttcagaagtactctttagttgatttgcttctctgtgcactcactcaccaa

SEQ ID NO: 18 - DNA, 501 bp, Bos taurus
  misc_feature (251)..(251): n is either A or G
aagcgacttagcagccccagcagcagcagcctcgtagcacctgttcattttccatgtgtcctcccacccttcattctgacctgttttaatggctgtgctgattcaagagggaagaggccacctttcttctagcttctgtggcttccagagccactcacatccttgccctttcatctgctcactctcccactgctctgtaaaaagatgctgactacacttacataactttcgccaagattaaaacatatggngttgctgatattcagtctatgatggctgtttcatatgatcttagcctaatatataccaggctaagatatatatattttatatacactatataaagtacagtatatttataatacatgtagacctgatttaagagcgactcagtgctattaatttatcagcattaatcataccttaggaagcattattatcatcatccttgttacatctgaggaaactgaggcaaagaaaggtcaactaacttgcccatag

SEQ ID NO: 19 - DNA, 501 bp, Bos taurus
  misc_feature (251)..(251): n is either A or G
gggaaagattgagagcaggaggagaagaaagagtgtcagtggatgagatggtttgattgcatcactgatgcaacagacgtgaacttgggcaaacttcaagagatgatgaaagacagggaggcctggcatgctgcagtccatggagtctcaaagagctggacactactgagtgactgatcaacaacaagaagctggtctcaataaaggatgttttgatttggtagagtggaagataatttgggaaattatantcctcatcaactcatttctaataagtgtatttattcatgtgactcctttgggtcaagcactgggatgagctgaccagtatgcatttttgacaatagagggaggcagggaagttttggccacctaggttgtgtggagggtgagaccgggaaaaaagtttgattaattttgttgtttgcccagtgcctggaaagaaaatatgctgcagccgttggtcaccaagctcttctttggaaggctacattctttagc

SEQ ID NO: 20 - DNA, 501 bp, Bos taurus
  misc_feature (251)..(251): n is either C or T
accagctcagtggacatgagtttgaacaaactctgggagatagtgaaggacaaggaagactggaaggctgcagttggacacaacttagcaactgaaaaatgcaacagagatataacactgaagtagaacgggattcatggaaggaagaatagagaaagtagtggaaaagctcctcggaagatagcatcagctctcacatggtttcacaggataattccatgaaaccctcaagaatcatggggactttattnctgagaatcccaattttacttacattactccagaatataggaaaataaggtaaaccttaaaattctaagaagtaaatgtaactttgagccacaaagttgacaaagattttatcaaacagaaagctacaggtgaatctcatgtatgaatatcaatacaaaatcctaatcaagtattaatgaaaacagcatctgacattttaaaaaattattttaaatatttatttacctatttggccacaccagcacatgg

SEQ ID NO: 21 - DNA, 501 bp, Bos taurus
  misc_feature (251)..(251): n is either C or T
tgagttctgtgcaaccttcatgcctaaacaaacatcaaacatggtgtttagtttgtctcatggccccgatgtgagttctgcgaagaagggacaggaccccttattcccagtgctgtcgaggatgctggccgactgctctagtcaggagctgtggctcccaggcctggctcttctgctgggatgtgctggcctccagtgttccagatgagggacagtgtccttagaccgcctgggctagtagcatcgattgnatgagctttacttgtaatgcacaatctatatttctgcaagttattgctgaaaagcacccacgttcctgagctgagtttcccttctttttttttttttctgtcttcagcctgacttggggacactggcgtcttccttggccacccagggcgtcccctccaaggctgaggtcagaggcaccattcccgccagggtgctgagtggaatgtctctcttggaggagaaagtcaacgactaggagtgaaattctgc

SEQ ID NO: 22 - DNA, 501 bp, Bos taurus
  misc_feature (251)..(251): n is either A or G
atgacaggttagctatatttctgctcagcaatgtaatattgaggtgactgcaaacaagtcacgctgctgaagtaaggagtattggctgagctgttactcttcaagttattttgtgacattggaattccatcaaatagagttaaatgtacctttaacttttaaacaagccccagttctgagccttaacctggatgatgacctacagattctcaacctgagttagaggtggatgaaaaggaagcagttttctngggcgtgttttcatgtcaatagattcatttactcaaatgtgtgataaacaccaactgctcacactttatgagagatacataaaaacgtggggacagaggaaccaattttgcaggaatttacagtctaagtaagcaaagagagtttaaactaagactcagtattttaattttaatacacacatacacatcagaccctagtaaaagtttctgcacagaagtgtgaaatctccaagtaatgttttccctacca

SEQ ID NO: 23 - DNA, 501 bp, Bos taurus
  misc_feature (251)..(251): n is either C or T
ttccctctgtgttatattgaagctacacccacctatcaggtcaagttcatctcacctaatacaggtgtgacataacaagaatatacatttgatctctacttcaggttcctgacacagagttcctaaatcccttgggatttccccagtcattgttctaatgaggtgatccttggtggctcctggataccttcaggatggtggctgctcaccaaatgattaagccatgatagcttagtttgggattttttttnctagccccataaacaattttccagggaggggagaggggctggagaatgaattaataaccaatcatgcctatgtgatgaaatttccatgaaaatcccaaaaccatagggttcggagagcttctgggtcgatgaacacatctacatcctgggaagatggtactcctgacctccaaggagcttcttttcccttgagctcttgcactcaagacccttctagattttgccctctgtaccccatcatctcgctgtt

SEQ ID NO: 24 - DNA, 501 bp, Bos taurus
  misc_feature (251)..(251): n is either A or G
cgggattccgacccaggcagcctggctccggagtctgttctcagacacccagccccatgctgaagggggccggattgctgggatcgctcagtgcccatcagcttgttactctagcctgctctcagaaagccctcataccagcccagttctaaaattctcaagtgaaagacagagtgttttcctggctgaggaaggagaccgtgtggcgaagagggaagagctgtgaggagtccaggaagtgacgtgtgccnactgtccagcacagacgtctgcgaggacgttgcagcatagttttggaagttctctggatcctaagcaaaaccacaacgaccagataaagtttttcctccatgggaaacatttttaactaaacaaaggcaacccccacatacagttcaaatgaaatccgcccacatggtgttggcatccgtgcaggaggaagcagtgaaagaacccaagcagtcagcagtgtcctgtgcaaaggcagcagcgcagtctgag

SEQ ID NO: 25 - DNA, 501 bp, Bos taurus
  misc_feature (251)..(251): n is either C or T
catcctactgtcccataaaattaatctttaaaatgtaaaaggacgagggtttttgttttgtttttcttagtgaataaagacaagtgcaaggaaaaaagttggatttgtgtcaattactaatcgcaacttattgatcattgacatctttaaatgtctgatcggatgccttagatttgtattagctgaaaaaacattagtgacgactgatgaagttgttttgcatttccatgcctggctcatagtacagactnaaaaacacaggcttcactcaagggacacagaatgatgaaaacctgaagggtgaaataagtctagctgaaaaaagcgttattcaagataagtagggagtgtctttgatgtgaaagatgcaggagggaagagaaaagtggtaaaaaaggttagttaatgaacacaccatgttcagtgccctgtgggagactgctcaaacataacttgcacaaaaatgaatccatctagtgaaggacaccaccatctctccag

SEQ ID NO: 26 - DNA, 471 bp, Bos taurus
  misc_feature (221)..(221): n is either A or C
agggatcaaattcgggtctcccgcattgcaggcagacactttaccatctgagccacctgggaagcccatttatagagatctctatactaaaggaagtatatatagacttgtatatgtatacattcatatacccatttgtacttaaacttttcttttctttttaaacagaacaccagctatattctgcagaatcaacgctatgggatataaaatgtactganttttttcttttcttctgaaaaacaatccttgctaagtttaacaagtttgagaatccctgtgttgtaaacattcttagtaaaagtttaactgagattgtaaatgtttaatttattgaaaattaaagaagcatcttaaaatattttttcttagtgtttaatgataaatcgtgtataataaagctgtaaataattatgttccctagattcttgaagctgttgatgctactcatatacaatgctctttagttat

SEQ ID NO: 27 - DNA, 501 bp, Bos taurus
  misc_feature (251)..(251): n is either C or T
aagaatggcatgagttgttccaaagcgaaaaaaaaaaaaaattcataatttgtttgttcaaagtttgaattatctagtaaagaattgttaattgacctgataaacatgttttaaaatgcagtcataatccagatatagcaagtaaatctatgatgaggttgagagacaagtccagaaaggattgatcttacattagtgaaatagtggttgaaaatgtggaagtggattgacttggtttgatttttgactcngatacttgctggccattttttattctttctgtcccttagtttttttaaaacttaattttattaattttttaaaacatttttatcttttaaatttttattttataaatggagggcccaatcacaatagagtccacaggttttcatttttgtataaaatggatagaaatagtaccttatctgattattttgagaatatcatattgattcttagagacattcttagaatctagtatacactcagaaaatgttt

SEQ ID NO: 28 - DNA, 501 bp, Bos taurus
  misc_feature (251)..(251): n is either C or T
tcttcggtcctttcagccctgcctccttctcctacttgcatctattaaccccctgcaggcaggctttggagttcacagttgttcaaactactctctgtcaagtgacaaacccagctgcctctcctcagtgtcccccacctccttcaatatgggcactgctaaccccttctgccccttcaactttctcccttgccttccagaacagcctgttctcattactcctccttgtgtcatctttgctgtatagatcnttcctctaaatcccaaggtacacggccctcctctctccaccgcttctgctcccttctgcaggatctcatcattctcatctgtaatgatcacctttctgtagataattcccaaatctatatacattgcttcagactttttctttggctttgtcccaattgcttactggacactttcataagtagagctttacaccagttcaagagcaaagtcatcatctgtatccaaatgaaaaagccccagatccccctc

SEQ ID NO: 29 - DNA, 501 bp, Bos taurus
  misc_feature (251)..(251): n is either A or C
agccttggcaggagatgcctgggcagctgtagttgggaggatgttcccagagccactgaattagtgcagggaggtagcagggaaggtccacatcactttcttgtcctgctcgtcatttcctactgggctggcaaccagtagcaagaaagcaggctagcccgcctgggcagaagcagggcagagaaagcggtgggccggacctggggaggcaaggaccagcacaaagaccaaatcccagctctctggactanagattccagctggaggaaacaagtcaacaaataggtaactattccccagctgggaataggtactgttccgaaagtaaaagggtgggctgggtggaaatcaaggagtagattaatcatgaccgttgcatttactatcgggaggtcacaggtcctctccaatggctcctatttgagtggagacagggcttcccacgtggctcagtggtaaaagaatccaatgcaggagcctcaagagatgtgggtttgatcc

SEQ ID NO: 30 - DNA, 101 bp, Bos taurus
  misc_feature (51)..(51): n is either G or T
gcttggggcttgtgctctcctagggcctggagaagccacttggagctccgnagccttcagactgcttcctcagccacctggcagtgaagcaggacagcctg

SEQ ID NO: 31 - DNA, 501 bp, Bos taurus
  misc_feature (251)..(251): n is either C or T
aaagagagtagactgtatagtttctttccataatctgttaccacagtgcccacctaagtgagacttcacctactcttaagagtaggtgtcagctccattttgcagaacagaaaactgaggctcagagacaggaaaggacttgctcaaggtgccacaggaagtgtcaaagcggggaggtgggctgactacaagcctctggctgtaccctgctgccaacaagaaagggtccaggccaagaattttggcaagangcaatgaagcgataaaggaagtcaacctctgatggactggtgggcctggctgcatgcctctggggcaggaggaaggtgttggagggggttggagggggttgtacaaacctgacctaacacagcccgcaagcgggaggtagggcttgctgagaaaaaagaggaagcaaaaacctaaacctaagggaggaagctgagtgggaacaaggcggtgcaggagctggggtccaggtggggcaagttgcggggggcg

SEQ ID NO: 32 - DNA, 501 bp, Bos taurus
  misc_feature (251)..(251): n is either A or G
atctgacttgcatggtctctgcctgtccattcagtggaggccacatgcctcatttgtgtcctttgacaccatctggttcccactccctcaccaacctgctttttcagggccatcattttgggttgaggaagtgtacgctttagctctgtgatgacaagatacatatcaactgctgcttttcctgcctgacaacttcctaggaagtccagagactaactttgggacaagaaggtttctctcctatcctttcngtactactaaagcctttgactgtatggatcacaataaactgtggaaaattctgaaagagatgagaataccagaccacctgacctgcctcgtgagaaacctatatgcaggtcaggaagcaacagttagaactggacatggaacaacagactggttccaaataggaaaaggagttcgtcaaggctgtatattgttaccctgcttatttaacttatatgcagagtacatcatgagaaacactgggctggaaga

SEQ ID NO: 33 - DNA, 501 bp, Bos taurus
  misc_feature (251)..(251): n is either G or T
tgttattattttaattaaggagaaacactttccacacataaaagattttcagaggacaatatgctttgtgcagttctggagggcagaaccaggaccaaaggatgggagccaaagttgaatacaaagaacttcatctggagtcttggaagaagggccaggctcctggggaggcgatgagcaaccatcactgagtactcaggtagagcagaggactgcttggaagggatgccatggaggggatgtactttaantaggaggtcattgtggtcacattttaagggctcttcttactctaagagccagtggttctcatccctgaggggagccttccccatgtgagttggatctctgtggtttcagaaactccaggatccattaagcccatatgcatttggttcagacacacccaggctttgggatttcttccaggcttgacctttgcagccatagaacttcactccatctccagcttgttcagctgcagcttacagatcagaggaa

SEQ ID NO: 34 - DNA, 501 bp, Bos taurus
  misc_feature (251)..(251): n is either A or C
tttaacctgcaccactgaaacccctctggtcaatgtcaaacacggccttggtgatgacacagctcctggatgcttttgtgtcctctccttgctctaccagtcagggtatttggcacacttggtaatccccaatccctgtcacaggccctttggcatccacgaagtctcatgctcctgaattttgttctatcccacaagctactccttctaagttttcttttctggcgtcagcatctttaggtgatatccanatatttaggagttcagggcccaatatttttctgtctatatttactcccatgagatttctttgactctctatgtattgatgcccaaatgtacatattcagccctgatcaagtccctgagatttaaattcttatatccaagtaattataatacgtcttcatgtctatttccaaaaaaaaaaaaaaaaagcattaccctgatttctctctgggccgtcagtcagactgatgatttagaactaaaatgatattc

SEQ ID NO: 35 - DNA, 501 bp, Bos taurus
  misc_feature (251)..(251): n is either A or G
aatttgtgaactgcatgtcaccagctatgctgtaagcccttaaaaaatggagacagtaggacagaatggtgaaaaattttagacttgaaagattggccacatggcctcagccgttttagtccttaatgcttactagctgagtggacatgagcaagtcatttaacttttctgaacctcaatttcagaatcattaaaatgataatatatatgcttagcattgttgttataaagatcagataagatttacagangaacattcttttaagtattgtaaagtatcacataagtatttggcctgttgttatatgtttaatgagtattcattaaataaatactggacacacaggaaacataagaaactagatctattgagtttatttgcaggaacatggatgttcttttaatttaccccccaaaaggaaactaaaataccagggttnnnnnnnnnnnnnnnnnnnnnnnnnnnnnnnnnnnnnnnnnnnnnnnnnnaaatcaacatga

SEQ ID NO: 36 - DNA, 501 bp, Bos taurus
  misc_feature (251)..(251): n is either A or C
aatacggctcttttgggtaacagagccttgaaaatgtgtcgggagagttggaggactggacataggattgttacctgaatccaggtactggagtaggactcatgtctcctaaaagaaatttaagggagaaaaaaactgccaccagctaattcttggagcctgtacatcccatcacatgcatgattcggaaggaaggagacttcaggctccacagtttgaccagcaaccaaaccaagtggcctgctgactcngtgactttattggtagtaacccaattatcagcccagggtatttctgaactgtccttaagagtttgtattctgaattggggtctggagcactaggtaagaggcagcttcagaactcctagaatgggaaatcctagcactgagtatggggttgtgttcagtgcttaaaatgtgcattaggatgaagatctttcattgtcaagatttaaaatttttaacagaatgaggaaagctgtcttctgagagctagagg

SEQ ID NO: 37 - DNA, 501 bp, Bos taurus
  misc_feature (251)..(251): n is either A or C
gaggggctggggggcatgctttaaaggtctcactagcatctcatttgctctttggagagttgggtttggcagcagagaggggagggcctctttggcttccaggaggaaatgttctcctcctctctttcctgttaaccttagtccagagtatggaggccaactcctccttgcgagggaagctttgatctgactaccaggcttggaccggcttgacctgagataatgttggataatgtaacaaacaagctacntgtaatatcagctggtcaatgagtagcctccccaaccccaaggcaaagcttgacattgctgttttaaaataataatctataatcatttatgtatatcttaagctcaaggcccaatagttcaaaaataaaaggtgagtacagagattcttctctgtacttctattttaatttgggggtgagggtcatttgctttcaaacctaggtgatgttctatatctattagggcccccaactgaataaagtattagac

SEQ ID NO: 38 - DNA, 501 bp, Bos taurus
  misc_feature (251)..(251): n is either C or G
attcaggtttgatttcctttaggattgactggtttgatctccctgatgtccaagggactctcaagagtcttctctggcatcttatattaatacaatgtgatctaatataattataacaatgacaacatataaccttttccatataaaagtgacatcacatactttggccatattcttttggtagaagaaggtcacatatcccacccacactcaaggggaggggattatacaggggtaagaatacaagaagntggggagcactggggatcacctgaccatttgggcatcatgcaagctgattggatcatcctcattccctttgacagtgaatagtttagtcatgagcatgtgacctactcctggctgatgggaagtaagaggataccttatagggtatgtggcagagactgacggcacttgcagtatgcacattcccctcttcttccagggcacagaattagacggtatttcctagcctttttacatcttagtagggttgta

SEQ ID NO: 39 - DNA, 501 bp, Bos taurus
  misc_feature (251)..(251): n is either A or G
caaatgtttccaaactgatttcataaccagatgatgcctaagatttaaacttggcaaccacaggtctattcttcagattgtgaaaccaatattcaaatagctgatgaagtggattccccatcgaatgacacaggttgcattgaagatgccgaccacagatttgtcatggacacttcatgatgtctcactcagcagtaggggtacaaaaccactgagaggattctgcttatggagcacaaatttcagtgccnggcacacagtaggcaatcaatagctatttgttaaacgaatgaatcaatagctatttgttaagtgaatgaaggaatttataacatactctctttccaaagtgaaagagaacgtattataaatgaggcttcaggcttctatccatcttgtccagtcttccattctgtatgtgtggaaagcgatgctgagagagcagaagtcatattcccaagaactcagtatttttataattgttcaaattatgtccccata

SEQ ID NO: 40 - DNA, 101 bp, Bos taurus
  misc_feature (51)..(51): n is either A or G
acagattcacccaaaatgacggatgtggagcctgtggtcaacaattttgcntcatcagcacgggcaggtcgccgaaatgctgtcccagacatccagggttc

SEQ ID NO: 41 - DNA, 501 bp, Bos taurus
  misc_feature (251)..(251): n is either C or T
taattcttttttttttttttcttttttggccacgctgcatggcttgcagcatcttagttccacaaccatgaattgaacccacgtcctcagcaaggaaaatatgaagttctaaccactggacaatcagggaattcccaaggatggactaattcttaaaacaaggttttgcagctcatactttgcctcctcacaggcccatggatacagtacggattgcagttgtgggggctggcgtgatggggctttctacngctgtgtgcatttccaaaatggtcccaggatgctccattacagtcatttcagacaaattcactcctgagaccacgagtgatgtggcagctggaatgcttattcctcctacttatccaggtgagagagggatttttgctcctctgtcagatatgatagatgtgagatagaatggtgttgatgttggttgactgccgtttacaaagacagctggacatttgtgtaagtgtaacgtgtgcctttgacatcttg

SEQ ID NO: 42 - DNA, 501 bp, Bos taurus
  misc_feature (251)..(251): n is either A or G
gagtattttcacttttacactattgactttttaaaacttgaatacattttttaaaggacagtttcagatttacagaaacattgtaaacatagtagagcgttcctgtacacttgtacccagtttcccctattattaacactttgcattaagatggcatgtttgttaaaattatatttcttgcccgagatctaaaatcatccttttttccaaggacccttggttccttttattagaaaccaatatctgggtgntagttggactcattgttacaactgttaacattttgggccttttgggatctgttctttatatcatagattgtttaacaccatccctttcctatgcagattagatgccaacagcaatcctccttctccagttatgaagatcaataatgtctccagatactgtcaaatatctcctaagggcacaaaatggttcctggttgagaaccactggtagnnnnnnnnnnnnnnnnnnnnnnnnnnnnnnnnnnnnnnn

SEQ ID NO: 43 - DNA, 501 bp, Bos taurus
  misc_feature (251)..(251): n is either A or G
gtattaagttattatcttacaacaatgaagtatttgtagtaacatcccaaggtcctataaaaatcagaactagaatttagatctaaatgaggttagcccaaactctttttccattgtcatcctggcccttcagatttattctcactgagggaaagggaagagaattgtccatggaaggcctcactgtgaaaggtggaccttggagaatggatgaggaaaactctgtcagatgggcatgggtgaggaacacnggaggcatcacacctcaccccaccttcccaggacttggtccagccctggaagtatgccccatgtcccctcgcagtcaaggtggatttcccctttgcctctggtattcctttctgcccttcatctgcttcactcttcccacccaagcagtgggaacatgatagtgagacaaaaagagaaaacactatcaggctatagaaagaattgatggagacatttttagataatattacttcatttaaaaaaatgtgt

SEQ ID NO: 44 - DNA, 501 bp, Bos taurus
  misc_feature (251)..(251): n is either A or C
gggaagtttgaagagctgtaaataagattgtaagtggttggaacacacagagaggagaatagtttgacatgagcctggagagagaagtgggcagagttagtctgtaaggactatgccagggttttgttctctgtcttgttaacaatgggaagctattagtgtgtttcaagaactggtgaaggggagtggagcagggctgtcatcaggcttacatctcagaaagaattgagggctgagaaggctctggctgntgtaggggaaggagctgagaagctgctgtggttgttgaaggggggtggggtggggggtaggggggatagggtgggaagtgatgatgcccggattaacattgcaacagagaagtaggttattcccacaggtacttagaaggcaaaatttgcaggactcggtgatagactgcctatgatacttacttgcgggtgtttggggtgggggttgtgaaaaattccagcatgacttcaatgcccttgttcacagaag

SEQ ID NO: 45 - DNA, 501 bp, Bos taurus
  misc_feature (251)..(251): n is either A or G
tgaggtttccattaggagtttgtaagatttaagccaaaatctcatccagaaaattttaaaatattcatatagtatgtaaagattcagagtgtgttattttttggagtgacctttttgacactgtttgcattatccacacaagattttataaaccagatcatcaaatctatagagtaaaaagaaaagagacattttcagttaccattggcatctattgaaatgcaaaggagaaagctacctactagaaaagngaattggctgatcttcccaagaatggagactatgttctatcaagggaaatatcctggatctgtctggtctctagaggaaaaagcgatctagatttgttaaacaatttaggaaatatatgttctgataatcctagattacttactgaatcttaaagttaattctggaaagaaatgacatttatttttcaaggtcagaacatgtgacattgctgtgtctgtctggtctggaatgtttaagtgtgtttcctta

SEQ ID NO: 46 - DNA, 501 bp, Bos taurus
  misc_feature (251)..(251): n is either C or T
cattttcataaatacacttactttcctcctttgaacaggcacacctgttttctctactaactctgttgcttcggtcacttatgccctagggaaagcagttgtaagaaatagcagatgcagaatttacacagcagagagcattccatcccttcagtttacaaagtctgcagaaaacaacccagtggtgcaggactattttatgaactgtgacctcaagccgctcattgaaaatgcagttcctatcaaattgntctttgcagattttcattttaaaatgatgaacacgtttggggttagttacacttgaaccggccacacttgcatcaacaaaaatcttcgcaaaaagtttacattgtgtacactattgcctgttttacagatggagaaactgaggcacaagtaccttaccccaaacttctgcaaaatgacacagaacaaatacaacctgcccatagcacgtgacaacatctaaaaaggaactaggtgtgttcctgctaagcg

SEQ ID NO: 47 - DNA, 101 bp, Bos taurus
  misc_feature (51)..(51): n is either A or G
gagagcgatcagcaggagtctctgcacaaactcttgacatccgggggcctnagcgaggatttccgttcccattatgctcaactccagtccaacatcattga

SEQ ID NO: 48 - DNA, 501 bp, Bos taurus
  misc_feature (251)..(251): n is either A or G
tctattatccttgcctggaaaatcccatggacagagaagcttggtgggctagagttcatagggcgctagagtcggacatgactgagtgactgagcatacataaacagtagctcttaatggctgtgaatactgagaggatgaaacccaagatgtcagctgtgttcaccatttggaagttttataaagcttgtagatatctttaaaatgtggaaattcaataagtgaggtttatttctggtcataaataaagntaattgaatgttgatgattttgcataatgcctattcatagcctttttcattttggagatcggtttgaaatagtgaaaactacggcttacaaggtcttggaatggcaagtgatcttgatgcaaagaggatgcttagatcagaaacctaggagatttatagaggcagagcctaaatctccattttctagcataaagtcatttgaactgctaagaacacattcaattcatttcaggaaaacaataggaatatt

SEQ ID NO: 49 - DNA, 501 bp, Bos taurus
  misc_feature (251)..(251): n is either A or C
agttctgattttgtttatttttcacttcacttctatcagttgtttattcatctatattaaatttcttatgttaggcacataccgatttaggatcaactgaccgtttttatcataaatatttctttttactggtaattttccttgtttcaaattctacttttttatgatattaatatagttacttaagctttttgaaattatgttttcatggcatgtccttttcttttttgccattttacctgtcaatatanttatatttcaaatggatttcttattcgtagcagagagccagggtctttctatccaatccaataattcatgtcattaaaataacatgtttaaaccatttacttttaatgtaagtatttatgtggctgaatttaattttgaatttttattgcttatatttatcttgtttgtttcttgtctgtttcacattttctacattcatttatattttttttagcatttggataattgtgttatcagtcagttcagtca

SEQ ID NO: 50 - DNA, 101 bp, Bos taurus
  misc_feature (51)..(51): n is either C or T
ggaacccaagtttgaagatgaccctcttccattttacttgttatatacaancacgtgttttctgtttactctggcttccgaacaccatcctcgctgtgacc

SEQ ID NO: 51 - DNA, 101 bp, Bos taurus
  misc_feature (51)..(51): n is either A or G
aagctggagaccggcctgtgaaagccacacaccttcagcaccaggcccccngggagtctctggtccctgcccgctcttacctgttcttgctgatgggtaca

SEQ ID NO: 52 - DNA, 101 bp, Bos taurus
  misc_feature (51)..(51): n is either C or T
gaggtcgctgtgctccttccttcgtggagcctcgggtgactgtccgccgtnaggacggaggccctgctgtaaacccgtcctggtgtctccgcaccacccca

SEQ ID NO: 53 - DNA, 501 bp, Bos taurus
  misc_feature (251)..(251): n is either G or T
tgtggcaaatcaataattttggcaatcgtattagctgcagatatccaacacatagacccacacactcctggaatttggcacagcttatctgtattttaggaagtcataactaaagctgcaaaaatatgttcgggttcactttatcaatccagggactgatgtattattttatatttcactctgaaaaaataaaattaaaaaaaaaactttagggtagaaaaatacatttcagaagaatctttacttcattnttaaattgcccatatctggctctatgctatttaacttattttagggtttttaccatggatagttagatctaagtgaaaaaagaattataccattccccagggtataatatacatgatagaaaataactgtccttctgtattcacatcgacacagacaattaggaaagggataatctgggatctaatttattttctgcagttgccataaaaagcaaatgtctattttcacttgggaaatgagatacatgat

SEQ ID NO: 54 - DNA, 501 bp, Bos taurus
  misc_feature (251)..(251): n is either A or G
aatgaactgtattcttttttaatcatctttttagaaccttagaaaggatttaatttagcttagttttgtatgactttcttagaattttttctgtgttacatggtatttaattttttattattgttacgttcaggacatatggaaaattattatctcttttctaagttgaaaccaaaagaatttttagcttcttaatttaaaagtgttctcgggtgtataacaagcttattgtaagtctgaactctttcctntttacctgtcattagaagaaaaaaaagaaaaaagtgatagcccaaaacgctcaaattttagtgtacatgggattaaccagaaatttgttaaaatgaagattctgatttagtgggtctggggtgcagcctagggttctgaatttctaataagttgccattttattctgatgcttgttctatagaccacacattagaaagtataatatgacctcaaactagtgaccctctggagagaaaacaggagaaagcg

SEQ ID NO: 55 - DNA, 501 bp, Bos taurus
  misc_feature (251)..(251): n is either C or T
ttcagatgtttgcattcccacctctcctaacactcccctggccccagcttaacttccagacacctccgtccatctgggtcttccagcagacacccacccctcatggaacagaggcaaatcattcctgtaataccctgcttgagttcctgccctacagaatccgtgagcaaaataaagcctgtggtggttgtgtttcaccagattggggtcatttgatatgcagaaggaaatgtatccttgtttattcattngggttgattccaatcttttgttaatacaaatgaagctgcaatgaataactttgtgcatagtttgtgtatatttagattcctagaaatgaggattactgtgtcaaagggtaaaatgcatacacaattttgttagctattgccaaattcctctctaaacaactttctttgtgcttacaatatatttgtacatgtataatttccctctccaacataactacagagaaacatacatagagaaaaatgcacaaat

SEQ ID NO: 56 - DNA, 501 bp, Bos taurus
  misc_feature (251)..(251): n is either A or G
gaagcagctgtgtctgcttgcagcttctagggcagcgcgccaggctgctgagaaagggcatgtttgtcactgatgggatgttctggaaccctccctcagtattgctgcaggatttcaagtctccctccagatgtgcaggtctatggctctgtggtccctggctcagcagcaaagagcaaaatgggaacaaggttgcagggaggagggctctagaacggtccaaactgggctttacaagactgtcaactcgngtggtattggtgatttgaatcaattggttctcagccctctacatcttagcaggaaggccttggctgacattttcgggaggagcctctttgaacaggaaagggtcatcctaattcctactccttttcactgtacagaggaagactgggagcctgaatcagaggcagataaatccagagagttacgggcctgacttctgagtcattttagaggggcaggcacccttgcactccatccactcccctcagacat

SEQ ID NO: 57 - DNA, 501 bp, Bos taurus
  misc_feature (251)..(251): n is either A or T
accctctttacacaaaaataagatgtgagaatcactttcatttcttcttagaagagtgatacctttgtttccctactctcccttcctcgaaaacaaaaatgaaagcaaaaaccacacaatttagagataagtcataaaaaataaaagtcatgtgttatttataaatcaatcatacagtaaaataattattttaaaaggtcacaaatacaacagttgcataaagtcagctttattttaagtgatcaaaaaanttttacctacttagtttttttgctgtcagagtgtactcagtgagttaaccaatgaattaaaattgctataagctgaaaaatactacgaagttgacaaatctctgacctcatcaacttacacatttacagtatcaggtgaacactgggtgcagaattcagttcaccacttcactaacaagcatgaaagatcttgatgatcagaattttcgtgctgtgtaaatttagcaggcaattttctgtgcactaaact

SEQ ID NO: 58 - DNA, 501 bp, Bos taurus
  misc_feature (251)..(251): n is either C or G
ggccagggtccctcgttagcaactggaggtttaaagcaggtaacttgacacatctcaaattagccttccttccttgtaaattgacgggcgaaatcgctccctcttgaaagtgcacggagaattaaaacacttagcatggtgtgttctaaagacagggaggtaccttttctaatgcacagaagccctgagtgtggctgtggattcagcctgactatgagtcctgatggctctaagtatggctgtaattgatngcttaaagtgatggtggtaatgatccttatcttttgaatgtttgtgatcagaactaacgatgagtaagtacaaacagtgatcttagacgagcaagtccctacccctggcttcagtccagtctgaacaccccaaggtcggcacagaaatgggctgcccagtgacccctctgctaagtaaggctgaccgcaccttcagtcacctgtccatctcttcagcaaacatctatctgctacatgcctatgctggacg

SEQ ID NO: 59 - DNA, 501 bp, Bos taurus
  misc_feature (251)..(251): n is either A or T
tacattttagtctcttacttcatgaaaagtgatggcaagcacttgttctcaatgtaaccttgatgcaggagaactcaggggccaattttcttacctgtagggtcagcttgtatgagatgggatggttcagtcctgtggttttcctggccttgactctcatagtctacactttgggtttccaaatacagtgactcaatttcctcatctgcaggatcagggaaagtaagagacccagcttcatagcacttggngggtacgtaatgagttaatctactcagagtggactttatgtgcccagcacagagtaaaccatccataaatattagccttattgctattttttgattttttttcctacaaagttgtgtaactgctatgtatcattctccctttccagctctgaggattagtgatgagtgagagaaataaagccccagacagaggtgacttgcaggcagtctgaacactcagggcagtgccaggtggggagtcagcaataaa

SEQ ID NO: 60 - DNA, 101 bp, Bos taurus
  misc_feature (51)..(51): n is either C or G
aggaaccattgcttctgtaccagccaatctgctgctgctgaagaatgttantgccatggggctgtactggggcagataccggcagcagaactttcccgtct

SEQ ID NO: 61 - DNA, 501 bp, Bos taurus
  misc_feature (251)..(251): n is either C or G
agagggatctagttccctgaccagggatcgaacctgggaccctgaattgggagcttggagttttagccactggaccatcaggaaactgttgataccttgtttaatgcagacagcaatgttgtgagtagataatcatacatattattatccctattttctagatggtgaatctgagacttagagagattaaatataactccagctccccagtgttgggcctgggcttgtttgccttcaaatccattccttgnccttcctctgcttagctctgtatcactcggatgccaacctctgcaggctgccaaaagagctgacttcctttatagatcgaccaatgggaggcagtgataggagcctagtaggcgggaagaagggagaagccaaggtttttctccctctctctgtctccggaaatagttccaggagcagctgcatttcctgtgtctctacctcccaccagacagtgccactgtgggtccagcttccacctggggagtccag

SEQ ID NO: 62 - DNA, 501 bp, Bos taurus
  misc_feature (251)..(251): n is either A or G
gggcgatcaggaaggacactgtgagggcggccctggatggatgtgtctttgtccaggtttctgaggggtgggtaaagtgaaaacaagccccctttgggtctctgcgacctgagggcccctctgttactctcactttccctctttcagcgttttcaaatctcagatgttttgttcagacgacgtgcctctggctctcctcatcaggaagttggaaatttcaggaaggaaatctggatcactgcatatcacanggaatggtgtatttttggatttcagaaagaatggggtctctggcaaggagtcactgagatcatgccttgcgaacaggaaggatgtatcgggtcacttacattgagccactggtgaacgtttaacgttagctgggtcttggggggaaaagccccaatttgtatgtagcgtttccctattcctgtggtgtaaatactcccaccagggcacactgcagggtaccaaatagctgtccttatacacagagctggg

SEQ ID NO: 63 - DNA, 501 bp, Bos taurus
  misc_feature (251)..(251): n is either A or G
tcatagattttataagaatcctacaacagaacacttacatatcttccttttcttttaaactattattataatatattttaggctcctattgctttcattattaactttatgtgtgaaggttcagggttaaaaaaatgcctctaatcacacagccaatggttaaatcctggctctattatttactcactatgggaccttagataaatcattttacttatctgttcctcagtagcattgtctacctatcatgngatgttcaggtgtgatgtgaggaatacatgaaacaaggcatgctgtgcaaggcccaccataactggcccaagtggagctggaggacatagcctcccttggggccaaacacaaaaaggctctcagagccgcaagtaaacagtttcctagggctgagcacaccctgctgcttggtcttcatgatctgactctctgtgacaccaggagacaccagcagatgccaacatgaatcttagagccaaacagagggag

SEQ ID NO: 64 - DNA, 101 bp, Bos taurus
  misc_feature (51)..(51): n is either C or T
ctcacctgcagcgccttctccttctacccacctgagctgaagctgcgcttnctgcggaacgggctggccattggctctggtgagatagacatgggccccaa

SEQ ID NO: 65 - DNA, 501 bp, Bos taurus
  misc_feature (251)..(251): n is either A or G
cttggtgggaacagcgggatcaaaatgctgaagtgtttttcgtcttctcctggttgctagactgtttctctgattgtgatgctgccctgagacaaggtcagggcaaagtggatgagttcagttgtgtttaggatacaacaatacatgacttctaggaccaccctgttttccaaccccaaatcacccaaatcccaggagacagcagaaaagagctctccctccatggaggcataagcaagcagttttcaccnagttctggctcaaacatcctgttgggaggggtgggctgagggaccactgcagccagcctataccgtccagctcgggttctctgaccctgacatactgcttcaggtcacatccactgaggcacacctagtggtcagatggtcttacagcagtaacggggtatctggactcaggggatctctcagtccctacacaacccgaaaacacacttctttgcatgaagctatgaacacaggagctcttgtctggact

SEQ ID NO: 66 - DNA, 501 bp, Bos taurus
  misc_feature (251)..(251): n is either A or G
gtctttgattttggtgaagtttcaaaattttaaatctctgctctgttctgcgtgtgcttagacccttaattaccagtgcattgccaggttaaattctcctcctcaagaccaagtttagatgggcaagccattctgctgatcatatccccgtcctttgttctaaacccactccttgttcttccagctccagaattccaaacctgcttaacccacatccttcacttggaaacaaaagaattttatcctagccntaagtatgaagaactgtgaagagctggggtcagttatggggaatgccatgaatatttaggacactaaataatatctttatcacagaatcattgacctgaatttaatatgtaagtttgtgatcttggaattttcttcacccatacaatgacgggatttacctaaattacccctgagatctctttcagtatgaaaattcagtggtaaatttcctgtgtcagacaggacagtttgcagtttgcagatttctgc

SEQ ID NO: 67 - DNA, 501 bp, Bos taurus
  misc_feature (251)..(251): n is either C or T
ccaacccagggatcaaacccaggtctccggtattgcaggcagattccttaccagctgagccacaagggaagtcaaagaatactggagagggtggcctatcccttctccagcggatcttcccgacccagggtctcctgcattgcaggcagattctttaccaactgaggtaccagggaagccccgatgtaaaactaaccttaatattcatactttgttgtgtcataggaaaaataaaactctaatattacttnctatttgtatgtttattccaggatagtgctaaaattgtaacttgaagctttttaaatttattactataatttaataagatttttaaaaacctcaagaaaatcaagggttttctgatacattgtgcagttaattgattaaataatgatgatgcataaactctctaaaatcttgaacttttaattcacttgacctatacagatcacctatacatagaactatgctgtattggggaatatcaatacatagcac

SEQ ID NO: 68 - DNA, 501 bp, Bos taurus
  misc_feature (251)..(251): n is either A or G
gacatatcagacaagcatttcagagtgccccaaatccccttcctctcaccctccttctctccctccttttccctctccttccttcactttttctttccttccatataagtatgaaaagtgagggaacaggaagcctgtagtttcctctatttacctacatttgtctattgcgtcttcttggaagggccaaacagtgctttgtgttaccagactatttcactttcaccgtgaaactcagttttctaaacacntctaacttgtctttctcttgtcaaacatggattcttgtctgtaagaatgaatgctctatacattctaggttcttatgttcataaaacacccatccaattttgtaacttttccagctttctggtacacgtgaacttctttgaaatttgctcattttatattggagttttttgcttgctttagtagagaaaagaacgtagcttgaattgcagtgagaaagataactctctcattggcagtgagtcagtttgt

SEQ ID NO: 69 - DNA, 501 bp, Bos taurus
  misc_feature (251)..(251): n is either G or T
tctctcatggtgaggattcttccggatgtttgatgagtctgggtttttttgtgctcatctcttttggggttgcactgggtcttctttgcaacatgcaggcttagttgccctgtagcatgtgagatcttagttctttgaccaaggattgaacccacatcctctgcattggaaggcaaattcttaaccactggaccaccagggaagtcccttgtgctcatctttatttggttaggaaactctcctccgtcagntgctggtgaatgcagctcttcctataaccagccccaaggaaaggggcgtggtctacttctgatgtggagctctggttttgttttgtggttgtggggagactgccctgcttctccctagggctgaagtctcttggggcatgacctcggctccctggacccaccctttagcccaggcacagtctcctgctattggctgcttgggcgaagctgggtaaagatggggatggccagcagggactcaatcccaggc

SEQ ID NO: 70 - DNA, 501 bp, Bos taurus
  misc_feature (251)..(251): n is either C or G
tcttcccaacccatggagggaacctgcatctcctgcattggcaggtgggttctttaccactgagccaccagggaaggccatactcccaactactacaggaatgagagagtgagttgaccaaattctcaaatatatttttttggtcaatagtcccctttcctaattttgaaaaagacagaccaaggggatgaataggaacccttgggaaaagccaaggaaaattcactgagtcatccttatacagcatcagncttaatcagtcaagatagaaacaggatagggttggagcagaagttatttctttatggagttataagacatgtggttgcctgatggccttgctcctggaaaggaaagcagctgctgcagggggagtgaccagtggtccaggacatttgtccaggaggagggatctgattccaatttttacagaaaggtttttttcttcttaatcagagaaaatagaaggtctggacagaaatgcccttgtttaagatgagt

SEQ ID NO: 71 - DNA, 501 bp, Bos taurus
  misc_feature (251)..(251): n is either A or G
ttgcagctagagttttggaatcacattaaggtgtccgtggggaagaagtgagattgggaaggctgctctgatgattcctgctggaaagcaaggtgatagtgatctgtttctgcagcaccttcctgctctccaaactccagctctgagagcatctagagtcagttgttgaaagcatctttcaggccagcttcgcagctatcatagtatgttcttttcccttataaacaactttgttggtggtatattttacntatcctgagatttatttattgcaggaatatacttcagtgatttttagtaaatttacatagtgatacaaccatcatcattaatcaattttagaacattttcagacccccagtaaaatgcccatgcccatttacaattaatctctctccctacccttagccccaggcaaccactcatatacttcttctccatacatttgtgtttgctggatattttatacaaatagaatctataacatgtggtctcttatgc

SEQ ID NO: 72 - DNA, 501 bp, Bos taurus
  misc_feature (251)..(251): n is either A or G
gtcagaattgtttgatcttcccactcagagctccaactgggacagctggacagaagcccaaatctatagttccattatcctggggctgaatcaaggctctccgcttaatagctatatggctctcccttaatcactgtacacttccttatatgtggggtggggaaaaggcctgatgagatcctttgtggaggccccttgacccctgagctccaccccatcccccaacaccaccagcaagggtaactgacccngtggttctgagccctcaaaatggattaagagaccctgaagaacaaatctcagatgggaggccaagctgtctgtgcacatggcaggcagccatctcttaaatgtagggtatgatgagctcaggaatctgccagggtttgcagagaggaagttgcctggggcagcaccctgcctccattcacaggagggccagaccccttaccgatgaccccgaagatgcccaggatgttgggggcaaagatgaccagcagg

SEQ ID NO: 73 - DNA, 501 bp, Bos taurus
  misc_feature (251)..(251): n is either A or G
agtcaaatgagataatccacataaaatacttcacaaagtgtcaagcatatatgaactaaataaattctgaataaataagcaataaacttcatcccttcaaccatctaactattactcattcattaattcactcagtaactcatatattctacaaatatttactaagaatctgataagtggcaatgattgaactagaatatatttgactgtggatacatctatcagatgaacaaaagcaaaaagaaaaaaangaatgaaaaatatctatctgtaatgccctcacagcctgggagttataagatacgagtattagcagttacagtattagactatgttgacaagactttgacttctgttttatttgaagaggcactgcatcgggaagcctttaaaaaatctttgggggaatgactgactgatggatggttgttaaagaaggttcaacttccaagtagggaggcttccaatgatggtgtgaaactgccccagttactgatgtcg

SEQ ID NO: 74 - DNA, 501 bp, Bos taurus
  misc_feature (251)..(251): n is either A or G
attaaaacattttagaggactacctgacttttgctctccacccgtaatttttaggagtcctcctttcacatatgaccctggagcagtaaggacttttatatttatacctaagagatcaaaacaaaagggtctgtcctcaagttcacagataagtctgcgcatccccagagtcattggccaacaaggtcagtatttgctgactgagtaaataacgaaaatatattgaaaaaattatttagaggcttattgantagtatggaagtaaagactcagaaatgaaaatgccaaccagcgggcagagggtccagatgacaacctgagatgctcagggtcacaaaactctttcagagaatagtagagcagacctaatactgcactgccatctcccctcaccctccagctcacttactcagtgattacagctgtgactttgacaaacaggcgattattttccatgccctggccctagtgaggaacaaggcattttacttgagggtctgt

SEQ ID NO: 75 - DNA, 501 bp, Bos taurus
  misc_feature (251)..(251): n is either C or T
ttttcccctcaattcccaaagaaacactgactcataatcacacacacacacacagtcatttggaaaaaattaatttaaaatctgtttttcttaagactttcaattatttcggccccatcactaagcattcagtttaaagaaaataaaaactctaggcaggcaaaatcatgaccaaagctatcattgtacagcatattgatggcatatctagaagtctgaaaccgagaagcaatatgaaaagttacttttgnatttagtgctcttttttttttagttttcaggtttgtagcctaagcattaagataaaaatgaccaggtggcagggccagctgattatactaaggcttccctgaagaactgcagctgcaagtaaattcttttctgaccagcctttattattggcagttatcacatagtatatgctgtccccctgtgctctcctgggacagtgggaaagacatggaaatgagaaggcagttggatgagggtggaagagatacc

SEQ ID NO: 76 - DNA, 101 bp, Bos taurus
  misc_feature (51)..(51): n is either A or G
ctcttgcacttggccaagttgatgccaactcagtggaggaccatcgctccnatcattggcagaacagccgcccagtgtttggaacactatgaatttcttct

SEQ ID NO: 77 - DNA, 501 bp, Bos taurus
  misc_feature (251)..(251): n is either A or G
tttgcctagaaatgcagcccatagcattcatgggtttactaaacagatgatttcaagatccctggttagacacctcccacctaccttcatgcaaatttgaaaagtacatccatttctcaagaaaactttacttttcctctcagaaagttatcacagtatgggggctttgctgacacaagatattttgttatcatgtgttagaatgtatgataagtcaacaaatcactggggtgtgaacagcctccccaggngttgtgaattttatttgggttcttcaattctaccttccattttgatactttacagttctgttttctgaatgacttttattaaatcactgagtttttcaattataccagtttctatgttttcccacttcaataaatttagttttaacacttattttgtacttaagaatcttgacactatttgtttatttcaacacctttaacatcttgctctattttttcccctattatttttatttcacagatatttgta

SEQ ID NO: 78 - DNA, 501 bp, Bos taurus
  misc_feature (251)..(251): n is either C or T
aagtggatgcaattatttttttttttggttcatacattgtaatgcaactaatattgagacaacacatttcagctcatggcattctcatttctctgtagtcagaagtttatatctttcaaagacctaaaagagaactatatgtacttacatatttatggggaatattacaattattgctagcaactaacaagtcaaggttgatgcagcaaacaatgttcctgtctccatgggccaaaagcaatagtagctangttaacagaaatgaaatctcaagtggtagtcacagtgaccttggctgtctggaacctcttatttctgacctttcattagattacttgaaaaaaacttataaatgtagactgcctccaatttttctaaattaataagccagtgaactgccattgcttttattgctcacaaaatttccagcggacatctaaaagcagtcattttttttccagttgaactttcagaaaccagaattaaattcagaagtcagag

SEQ ID NO: 79 - DNA, 501 bp, Bos taurus
  misc_feature (251)..(251): n is either C or T
atttagaggctaaatagatagtgaaaggcagcaatagatgaaggaaaaacctaccagactaaacacttagggagagctcacctcaggacacctgctgccacgtacaggatgtcttaatcaagaagcatttctcagaaagacagggctcttgcttgactttgttatagatagatagataatgaggtcaagcaagagtcctgttaagataagcattttataggtgagaaaaaagaggctcagaaaggttactnaacttgcccgaggtcacacagccagaaacgctgagtaggaatgtgaatcctggtcagcctggttttaaagccccatgccctttatatgatgtgaaggatacaagctgagaactgattttaaaagctgtgttcaaggtatgttctagattagtatacatatccaaatcattcttgtatttaaagtatctacatttctatcaagaacattaacatctacacctgcaacatggtccctcgaggcagtccctaa

SEQ ID NO: 80 - DNA, 90 bp, Bos taurus
  misc_feature (51)..(51): n is either C or T
gccgggatgcggttctgtgaagcgagtgccaaggataacttcaacgtagangagatcttcctgaagcttgtcgatgacatcctgaaaaag

SEQ ID NO: 81 - DNA, 501 bp, Bos taurus
  misc_feature (251)..(251): n is either A or G
tgtaccataaattatcagacatttttatctgccttgttcattactgtggtgctagtactgtgcatgctgcctggctcactacaggtagtgagtacatatatgttggatgtgcaaatgagagaagacataagaaaaggaagtgagttataccctgtgatgtgacccggaatctcacttcacatgtttacactcactgggggcacacagataccatccctgaatggcatacactctcttctccagcctactcngctacacctcatgtctgactccgctggccactctccggtctttgaaactcaccttttccttggttttcatggaatgctttcttctctgtttcttttccacctttcagaccgtccagctcaggctactcttaaaaaaaaatttttttttttttaatttatctggctgtggcaggtcttagttgcagcatgtgggatttagttccctgtctagggatcaacagtgacggcaacggcaccccactccagtact

SEQ ID NO: 82 - DNA, 501 bp, Bos taurus
  misc_feature (251)..(251): n is either A or G
tttaaggagtaatcaagagaagttccatggagaacacaaaaaaggtcaaattctgaaagatggtgatggagtgaagtctccccaaacataacacgtttcatccttcatctgtccagccccttcttatctctgcttcactttcaaattggtttgtcttggtcagtgactttgtgatggaaaacaataggacttaatgttagaaaggtagacaggagagtaattttgtggggttctaaatgtccctataaagngtgtaaattgtctgtcatttgagaaggaccattttaaggttttgaggagaaaagtaacaccaaaagttataattcagtaagatgaatttagtagaaaagtatgacattatatttaacaaaggggattagatataaagaatggccaattaaaaacctgttgaaattaactaagtataaagtttaaaaagtagaggggagggtcaagtagattctctggccttagaactgtgctttcctttccgttctttat

SEQ ID NO: 83 - DNA, 501 bp, Bos taurus
  misc_feature (251)..(251): n is either C or T
ggaaagcaggacattatttaaagttgaagcttgtgctctaggtggagggggtgtagataagtcagaattcaggcatatgtagatgtttttcatctgtagatgtttcatatgtagatgtttctacatccctggaaccatatccagggatgcattccagtgaaggtttcttcttttgagtggtaaggcatcattaaaaggctgctagtgggcttccctggtggctcagacagtaaagaatctgctggtaatgnaggatacctgggttgggaaaatcccctggagaagggaatgctacccacttcagtattcttgcctggagagttccacagacagaggagactgggaggtgggtacagtccatggggtcgcaaaaaactggacatgactgagcaactaacactttcgtttttttctctttcagtggaaataaaatctggtgcagtcattttgactctcttgtccttctcttcccattgtctctggacatgtcccaatcagatt

SEQ ID NO: 84 - DNA, 501 bp, Bos taurus
  misc_feature (251)..(251): n is either G or T
tgtccacccagtctctctttctccaatattgcacccatccaatctatccaattctttcaaactgaaatgctcatcaggcctctccctggtcccttcagtttggtttgcaaggccctccgtgataggatgccagcttagtatctggcctcatctccctcccctgacttccattccatatctttgatgggctattccttctcatgcctgtaagctgccacatgtgtcaatccttacatagggatttctacatntctacatagcacattttctactttttttttttcacctgaaaactcttatctatccctttttgtttactaaggacataatatgctacctcttcttggaagtccttgctaatatcattacctcgccccactatcaactggctaattcctctcctctgtatgtattgcacttaacgagttgtattttaactatttattcccccttaaactgtaagccatttgagaggaggactagttctatattctcagctcc

SEQ ID NO: 85 - DNA, 501 bp, Bos taurus
  misc_feature (251)..(251): n is either A or G
aatatactgacttcaggaaaatagaaatacattttcttccaaaccactcttcctgatttgcaaacccaagagagtttaagggaaatgcaatccacatgtttctggctatttctagtcacacacatttatctaaagggttcctttttttttttcctttgtgaaaataatttgatgtctatgaagctttgaaaataaatctggatactttttatttttctaagaaagaggaattttctttggataagagtaanttaatagaaactttttaagatttgctttggcttcaaatttaaaagtcatttggtaagggtggcttctatactcacttctaatctatcacaccatctagtcccttctctgtttttattatttccattacactgtttcaaattctttgtgtatagtaagtcattcattcacccaacttatatttagagtgcccttgggtgctggagacacaaacaatacggttgatgtgtctgccctcgtggagcttagagt

SEQ ID NO: 86 - DNA, 101 bp, Bos taurus
  misc_feature (51)..(51): n is either C or T
tctgcacttcactcggtaatattagcaaatctccaaatgttagccacattngtttgtttcccttgtatgttgtttattcatgatacttcaatgctgtaact

SEQ ID NO: 87 - DNA, 501 bp, Bos taurus
  misc_feature (251)..(251): n is either C or T
aacctcagatatgcaggtgacatcactcttatggcagaaagtgaagaagaactaaagagcctcttgatgaaagtgcaagaggagagtgaaaaagttggcttaaagctcaacattcagaaaactaagatcatggtatccagtcccatcacttcatggcaaatagacggggaaacagtggaaacagtgacagactattttcttgggctccaaaatcactgcagatggtgactgcagccttgaaaattaaagangtttgttccttggaagaaaagctatgactaacctagatagcatattaaaaagcagagacactacttaaatgacaaaggcccatctagtcaaaggtatgttttttccagtagtcatgtatggatgtgagagttggactataaagaaagctgagctccgaaaaattgatgcttttgaactgtggtgttggagaagactcttgacagtctcttggactgaaaggagatccaaccagtccatcctaaaggaaag

SEQ ID NO: 88 - DNA, 501 bp, Bos taurus
  misc_feature (251)..(251): n is either A or G
acaatttaggaaaaattgtagaatacctaaaagtatcaatcagtaaaagccattcataacagaagctatgagatgaaggcatgaagtttaactgaagacagtaaaacaaaacaagccaagacagaagggtagatttaataaagataaaagcagtatgtaatcaactataaataagaaaaatggccgaactaataaataaaccaaacaccaaatctctgagcaaaccaataaaccagaaatgcagaagtcgnaagcacaaattcataaaataataaatgatggaggaaaaatcatgatatttggaggaaattaaaagattgacaagaggctatgctgttcaactatgcaaaagtaattgaagacccagtgaaaaaaggaatatttttaaaaattttcaatacattaaattaattccaacacaaacagaatatttatagaccattcactatggaaaaactgagttgtgaaaatgctaatcctatgtcaaaaatctaactcaga

SEQ ID NO: 89 - DNA, 501 bp, Bos taurus
  misc_feature (251)..(251): n is either C or T
gatggataacacccacaagataaaagttctttgggacccttaataatttttaagtgtgtaaaagtgtcctgaggcccaatggcttaagaactgctgacacagaatttatgaggagaccaatgataaagggcagagcctggggagctgggcagccctggacggactggacaaagcgaggggtggtgtccacgttccccgggcaggctgggctgcaagcgtccctgggcagagactcaaactcggccaccacngggaaaggtgcaggctccacttccgcctcagacagacagagcaggacgcagggggaggcccagtgccgcctgtgacgtgaggggaggtctgggatgtccccagagggcacccccagggcgtggggggcgggagaggccgaggcagcagaagccacaggagaggcgaagagacggggtccgcttgggcttctgaccaagcgaccacagagcggggcttcccgcgacccctcctctgatgggattgatttgc

SEQ ID NO: 90 - DNA, 501 bp, Bos taurus
  misc_feature (251)..(251): n is either A or G
atcctctgtcatccccttctcctctcgccttcaatctttcccagcatcagggtcttttcagaagtagctggaaaaattatgatctgttcaggagtcaaaaattagatttaaaagctttccacagactagctgatcatatgaagtgtgggtgggagaggcaccaatgcagccagagttcatgtaactatattttcaaattggttagaaggatcatatacatacctgtaaaatcctacagtcaagaccacatnaaatgaatgccagataagtgttaaacacaacgagaattgcaggcacatagcagagagagcaagagagacagaaagagagaaagaaagcatgcacactttcagctgtgatggaaacctgcagattggtaaccactgaaaagaaggggagaaagtttatattttccttcctgaagtatgcatagctgatttgaaacatcccaaatctgcaaaacatcttttccttacggacttaaactggaagtattctccc

SEQ ID NO: 91 - DNA, 501 bp, Bos taurus
  misc_feature (251)..(251): n is either G or T
tgaattaagaaactgcaagagcaacatctagccatccatacttccccagagcgaagacaatcttcttctctcagatacaaaaaataatcagagaattaagtaataattaaatgagctagaatttcactgataggcattatagtggctgatattaagttactctctaccagtcaagatctctctactctgtgccacctgaacaaacactcctcacccgctgggaggtgttcaagcagctacacaggagaaanaaagaaaatgaccagtacaagaaaggccaggagtgtattgcgaggcagcatcgactaccttacaaaatcagaaaagaagaatttttttccctagatgctataatttcacttattaaatatctcctttagagatcaatagtactttaaggaatagaaaaattagagtgcttcttataattcaaggtcttaacattaaacgtaagtttagcataattttaagaaaggaaaaaaatagagatgtttacctgac

SEQ ID NO: 92 - DNA, 501 bp, Bos taurus
  misc_feature (251)..(251): n is either A or G
agaatacaggtttagtctagagaatgcagctaagggaaacaaatgcttaaaataatccacaattccatcatcccgagataacactgggaacattttgagatatacacactcaaaatttttaaatatacaatgcatatattttaaatgcagatatatgatccatgcttgtaaacttctgtactaaaagtcatacacattttagatgccaataaaaacatttctccaaaattataggtcgtggaacatagatntgtgtgtgtgtatcaccatttactcggagaaggcaatggcaccccactccagtactcttgcctggaaaatcccatggacggaggaacctggtaggctgcagtccatggggtcgctaagagtcggacacgactgagcgacttcgcttttacttttcactttcatgcattggagaaggaaatggcaacccactccgatgttcttgcctggagaatcccaaggacggcagagcctggtgggctgccgtctatg

SEQ ID NO: 93 - DNA, 501 bp, Bos taurus
  misc_feature (251)..(251): n is either C or T
gaagacttcccttttggtcactccttaaaggtgttgctagaacaactcagcaaggctcagatttggggaagttagtcaatctgctgaaaatggatacactggaagccaacattcagaagaaccatttcactgaagtcactcagccaaactaatatgaatcaagtagaaggtttataatgactgatattaaaaagacttcagcagccttggaaaatgttctagagtcttcagatctttcactgtctctcatncttgaccctttctgtcctccagcgtccatcacctctgacattcattccttgtcttcctcggtatcccctgcttccctgcccattagtatctttaacccctccgagatgtgaagatgagcacaccaacttcctttctccattccttagcagggatgcctgttttccaacctcagcaacttgtaactgaatccccataaagccaagacaagaaaaaacaaaaaacggggtttccagtcaatttacattcaac

SEQ ID NO: 94 - DNA, 501 bp, Bos taurus
  misc_feature (251)..(251): n is either C or T
ttgtgtcattagttcagcctctgaagcatgagactctaggttcggttttcagaaatgttggctcatagctttaggagataagattgtccttctgggtagatatagaagctttcagaccattatgtagagctcaaccgtcataatgaatttgaagccctcaactagcctttttattttctctaccgaagaagggcaattaggagcaactagccaatctcattatacagtaagtcccctacatatgaagtttngagctttcaaagatgagaagctgcccctgtatgccagtcattgtaccactgtacttttcaagatactttagtataggattttaaatgttctctttattttttgtttgtctgttttttatgtattatctgtatgaaaagtattataaacctatgaaagcatacagtactatatagctaattgtgttagctcggtacctaggctaactttgttggatttacaaatgtgctttcagaatggaacttgttcaga

SEQ ID NO: 95 - DNA, 501 bp, Bos taurus
  misc_feature (251)..(251): n is either A or T
tttgtgtggttgctgctggctagaagggtctggtcactaggtggctgacttttaaaccctaaaaggccctgggtctagtgctaactcactggtgggtgaagacagggttctgaagaagattctggggctgttgctcacccacaggtaagctagtgccagactactgttggcaggcagagctgggtccttgagtctggctgcagggctcagggatcctacagctagtatcagatcactgagggtgggataangaggagggggctggttcctagcacacatttgggtatgaggcctggggtatcctgaagcttttttttggcctgctagtggaaagggccagggcccagctggtcccagggcaggatctggcctgctctgggtggactgggtctgcaggctgtgggattgtggttttcttgtgcctggtgtctgccccctggtgagtgaggctgatccagaggctagtttaggcttcctggagggcagggctggtacctggcc

SEQ ID NO: 96 - DNA, 501 bp, Bos taurus
  misc_feature (251)..(251): n is either C or G
catgggcacctctgttactttatttaccttaactctctgttattacaattcctatttcacaagtgagaaaaataagattcatcaaggtgaagtagtttgcttaactgtaaagtgggagatgctgagccagcacatgaagtgttaagatcccttcagcaacttcatggagttcttagactcctgttttttttaagtatagacaaaaagatacagaggaacactaagggaggaatacaaagggatgagctcanggggtaggggtgttgtgggaatatctcaagggaaacagcctaagggagaacagctgactactaggtgattagtaactaatgtttgtgaacaaaactgggattctgacctgaaaagatcactgttaggactcaaagtcctgagacagacctcagcctaagggtaagaatggaagtagaatagaccagcaagtagaatcttagtttaccacagatagttgagtaaacttatatatcagccatttgcctaaaa

SEQ ID NO: 97 - DNA, 501 bp, Bos taurus
  misc_feature (251)..(251): n is either A or G
ctgcaaaaagtcggacacaacttaacaactgaacaacagcagcaattacctctgatgtagtgtttcacagaaagagaatggtgcccaaaacaaatataattgttaagatgaacacatagcttgagtcctcatagggttgtactaaattagatgaaacttttttagatgttggaggagatgttacatttaagtttttcctatagcatatttattttattgggatttcccacactccctagtgtgtccccttntcagatcaaataaggaaaatgagtctgccgtctttaacagctgcagatgtcctgtgttctcttccttaggtgttcttggtgaggaaggtcaaggggtcagatgctggacagctctacgcaatgaaggttcttaagaaggccaccttgaaaggtgaggggttgcagagggcaagacgcgcctcactgtggttgaccctctgcttgttatgagggagtatagccccccttttagtgttgcttgactcctgta

SEQ ID NO: 98 - DNA, 501 bp, Bos taurus
  misc_feature (251)..(251): n is either C or T
gaacatggaaatgttatgatttttcacatagtatttttagctttcttgttatttattcactaagtcatgtctgactcttatgcaaccccatggactatagtccaccaggctcctctgtccatgggatattccaggcaagaatactggagtgggttaccatttctttctccaggggatctttctgacccggggattgaggggttttaattgtcaaagttaagattgtgctatacttcaatgattactaactncaaagatgtataaatgcaccccaacaaattattcaagcaagaacttcctcattaaaaaaaataattttcaaaaaaattttctgtcagtgaatgcattgtaagcatggcttctgaggaaataccagagcattaatacccaaaattttccttcaaaaagtctagtcttcacagctatttgaagccacttacctcctagggctccctaatcttacctccctggcatcaaagaactacaaaagaaggatcatct

SEQ ID NO: 99 - DNA, 501 bp, Bos taurus
  misc_feature (251)..(251): n is either C or T
ccctgtgaaaccaaaaactcctgtgatactcgctttatctcggtggtctggaactaagccccaaatacctgtgaggtgagcctgtgttaacttctggggagttgaggcaggtcacctcagatgcttgggtatcaggctgcttattgaccaggtgaggttataataataatcatttctacttcctaggcaggttttagagacttcattctataaatgcttttgagagctatttttaaatgtgtgatttccangcttaggtcactacatactagcatgctctttttttttttttaaacgagtgtgaagtcctctccctgctcctgatggtttaatgactggagcgctgatgaacaggttgaggcaaacgcctattaatgttactctccgattaccgtgggttaaggaaccgaggctttggcctttcttggctgtttccaaatgagtcacaccttcccattgttcatgtaggtcagtaagttacgtagtgtgacctacgtaaag

SEQ ID NO: 100 - DNA, 501 bp, Bos taurus
  misc_feature (251)..(251): n is either G or T
ctttccttcactccagtggcatgtgtatgctttgctccaaggccctttcagccctcacatctgtccgcgtaggcccttgaactcactgtgcagcaaactgcacctgtgtgcttctctcttgtgccagcacagggctgggcgtacaggagctcactagatacttgaggttttccttttccataggacgagagaacagggaggaaaattagaaatggggaaagttaaatcctgtgcttattcgctcagccatntctgactctgtgaccccatggactgtagcccgccaggctcctctgtccatggggattccccaggcaagaatactggagtgggttgccatgtcctcctccaggggatcttcccaacccagggatcgaatccaggtctcccacattgtgggcgggttctttaccatctgagtcaccagggaaaccctaaatgcaaaatacagagagaaatgcttatgtcagaagtttaggtcagcaggctgaggacttgaga

SEQ ID NO: 101 - DNA, 501 bp, Bos taurus
  misc_feature (251)..(251): n is either A or T
ccttggtagcatggggggatgctgacgacgtggcagaactgagaacggatacaaaggtggagctggaaactgattaaagcttaaggttaaaaaataaaagttgatgattacatctgcatgttatgaggtgaggaacctatatatttaagcgctgacagacagaaaaggcacatttttactttttcactatgtcaaatgatagacttgtcttttcctgagtccccacattattttagctgttttagcaaacnacatttgagtctctgggccagaagcttctatgaccgtggcatctgcaaataattgataacttaacgtgccccctcaagaggtaaggtcaattcaagcagtacctgcatgtgtctgtcaccttctccaatcatgtttctccattccatgagtgaccgctgcaggtgctcaaggccggtttcccaaagccggacacggcctcccatatcaacagtgacaacgccacctctgccccactcggccagcagtgcg

SEQ ID NO: 102 - DNA, 501 bp, Bos taurus
  misc_feature (251)..(251): n is either C or G
atcatattttccattctgttggtttactgggacttaaaaaaaattgaatattatgttcaaagttctggaatggtccctataagaaggtagagtcatggagcaacttacaatttattggagaggcacattaagtcaaatactaaatgcactgtgatgggtacaagaacgagggacacagcatagaggaggggaggggaggaaggaacactccagaagcttttagaagaaaacagcaaaaacgggtcaagacngagtagatgagtcaatacagcctggcttcctataggctgaacctaacttaaatgtaggattagtttggcttgcaaatgtattcccaggatttccctggtggcatgtacagtgaataagaatttgcctgccaatgcaggggacaggggtttgatccctggttcaggaagattccagatgccaaaaagcaactgagcccatgaggacaattactgagcccactctctggagccctagagtcacaactaccga

SEQ ID NO: 103 - DNA, 501 bp, Bos taurus
  misc_feature (251)..(251): n is either C or T
acactgtttccactgtttccccacctatttcccatgaagtgatgggactggatgccatgatcttcattttctgaatgttgagctttaagccaactttttcagtctccactttcactttcatcaagaggctttttagttcctcttcattttctgccataagacccataacaccccaaaactcactactggccacttcattgcactccagagagaagagatccagctccacccaccagaatacagacgcaagnttctctaaccagaaaaccttgacaggatgggagcaacatccacaatacagagtaaccacaaattttcagcctacagaaaggccaccccaaacacagcaatctaaacaaaatgaaaactcagagaaatattcagcaggtaaaggaatatgataaatgcccaccaaaccaaacaaaagaggatgagatagggagtctacctgaaaaagaattcagaatgatagtaaagatgatccaaaatcttgaaaacaaa

SEQ ID NO: 104 - DNA, 501 bp, Bos taurus
  misc_feature (251)..(251): n is either A or G
ctatgggcaattgttcagggatacaataatcagtgactggtgtatcagacagaaaaacaagttatctatttgcaactgatgaaacagtgagttaaccaagactagtttggtagagcttgtggtatttatacttagtcacttaaatatttatttattgaatatgtactacatggtaggagtacaacagtgaataaatagacatgtctcaataccatggagattaccttctggttgggtatttctaagaaggnaacatttaagctgagccctaaagaactaggaagaggcagtcttacacaagagaaggggggttccatggagagaaaacggcatgcacaaacccctggggttgggatgaatgtggtcctctgtaagtgcggtcgaagtccagtgtgactgagattcccctggtggtccagtggctaagactccacactcccagtgcagggggtctgggttcaatccccagtcagggaactagatcccacatgcagcaactaa

SEQ ID NO: 105 - DNA, 501 bp, Bos taurus
  misc_feature (251)..(251): n is either A or C
ctgttttaacagctacaaaaaggagcagtgagactacaatcagggacatattaattgttctttgtccttcatgggggaaaggcctgttagtcacatataaatatccttcagctgtgtgagaacagccatgtagcatttacttcacaaggataaccatggccctttctagaagagctgcccattccctttgcagcccatctcttcctttcttcctatagttcagtatttgcttcatgaggagattacactgnggtaatacattctgaacgttgaaacacattttgggcttctctgggggagacatggatttctggctgtccttcactttcctcttttgttctttgtacagtgaagctccagggagctttctctttttttccaccagggtttcactgtcctgaagcattcaggggttggaaacccaacagactagtgagccagtgagaggcgttggaccacggtcagacccaacccaatgacatcaacggagacaatctcagt

SEQ ID NO: 106 - DNA, 501 bp, Bos taurus
  misc_feature (251)..(251): n is either C or T
agatcccacatgcctcagagcaactaaacccgagtaccacaactactgaagccatgctctagagcccaggagccgcaactactgaagccatgtgcctagagcccctgctcgcaacaagagaagccaccgcaatgagaagcctgagcaccacggctagagagtggcccccacacaccacgactagaggaagcccacacacagtgacagagccccaaggcagccaaaaataactaaataaaaactacaaaaanaaatcaatgaagccctaagatggataaatctagagaatatcatgcttagtgaaataaggcagaaagagacaaatacagtattatatcacttacacatagaatctaacaaataaaacaaacatatatatatgcaaaacagaaagagacttgtgataaaggaaacaaactagtggttaccggtggaaagagggaagggggaggggcaaggtaagagaatgggattaaaaaatgcaaactactgtgtataaaa

SEQ ID NO: 107 - DNA, 501 bp, Bos taurus
  misc_feature (251)..(251): n is either G or T
agcttaaaaacaattacagacagcaccatgactaatgaatactgcacagttttctttgtcagaaaaatgaatccactttgctgttcatccttagccttgacatagtgatatatagcttgtttcctgaagtagatttaatggtgtagattattttgcagacttgaaaatagcatggaaaatcagtatattgcttcatgtcatactgacatggctttatgtcttgccaccaaagcagaattggagcattagantatagtcaaaatatggaaatgcttgactctttgatttttgtctctatggttaagtgcagttgggtagggtgtgagggggggtaaatggaaaacaatcaatataattactggagttctaattattcttaggaaaaggaaaatcttttaaacaaaacaagctgaagacggttttaaaagtaaaaaaatattgaaatcatagctgtcactttaaagcatctttttttaggagaggataatcaatgaattcata

SEQ ID NO: 108 - DNA, 501 bp, Bos taurus
  misc_feature (251)..(251): n is either C or T
ttactggtaatcaagtacccggaggtaggctaggagatcatcagggaggaaaggtttcacagtaccagagtcacactcacgaagttctctacatccaacttctctttatcccaaacaaagcaaagctcacaaggaaagcaactcttaggaatgcccactcaaagactttcagttcagttcagtcgctcagtcgtgtccggctccttgcgaccccgtgaactgcagcacgccaggcttccctgtccatcacngatacccggagcttgcccaaattcatgtccatcaagtccgtgatgccaaagaccttgcatcatgcttaaagaaagaattaggggatccagttattttactactgagattcttttttcatcattaaagtgactaagattttaaattttattgactatcatgagtaagcaaaataatatatgcaaagcacttgtcacactacctaacacagggacaacctgaaatttttaatcatgatttagtttgtttgta

SEQ ID NO: 109 - DNA, 501 bp, Bos taurus
  misc_feature (251)..(251): n is either C or T
aatgttcttcttcagttttaattttctctggttatctatgtggggctttgttacttttgattccctttgtagtatttttgatagggggacaaagaacctgcttcttccccagtttgttatttgcctttagtatttttatggaattttctgatttctataatggcaaaattacagataccttcttttgtggtttctgcctttgatttcctggttagaaaattctcacctatccaaagactatataaaagttnatctgtttgtttcctggtacgtttttatttttttcacattagtgtctttaatccaaagtcagaagacaatggcctgtgggctaaatctggccctatttttgcaacaaaattttattgaagctcagccatgcccatgtgtttaggtgtgtttatggctacttcttgattccgtgacagaggtgagtagttgcaacagaggcaatctggcctgcagaccaaaatctgttgtttggctctgaatagaaaatgt

SEQ ID NO: 110 - DNA, 501 bp, Bos taurus
  misc_feature (251)..(251): n is either C or T
tctcaatctggaccaaaggagcatgattctgcatgtcccaaggggaagatcccgcatgctcaacttaagactcaaggcagccaaaaaacaaaataaataaactttgttttaatagaaagaggtaatagaaaactaggaaagatgtcctcaacagggtaaagaaacttttcatatatatatatatgagtataagttagatacatttttgtcacatttcacacacatatatggctgtgctatgttttcacttnactttttttaatttcaattttttaatctatgtaacattttaattaaaaattaaaaaacagaatttttcttttcttaaatgttaaaatattctcacaacagaattttaaatgttcttgaaaaaacaaaaattcacacttccgtgggagaagaaaaagatctaaattatactttaaataatccagtatccctaaactctttcattgccctacaccatatcctaaagagatttctgcacctgaacatcattct

SEQ ID NO: 111 - DNA, 501 bp, Bos taurus
  misc_feature (251)..(251): n is either C or T
tgggtccccagaaacttgtgttcctgacttatactgcaaataaaaagatattggtttccttgaacttctgtctctcagagctggatatctacacgtttgttgccagaactccctaattgcacatattttcttgtatttcttttctttttctgtttgcttttatgtctactgaccacaataaagaacccacttagattaagattttgtatttttggcaacatttaatatactttattcaggcatgattttangtcttactccataaaacagtccaaacccctgtcattaccctggctgagttaatttcttcatttgggctaccagaaaattttgtacttctattggacacatcctacttgaaaatttcctaggatccccataaaaccatgaactctttaagaaaggttatttatttttatctcgccaccatcagcacccaaattcatgcctgaacaagtatatattatctgagtggccatataagattattactctcaaggt

SEQ ID NO: 112 - DNA, 453 bp, Bos taurus
  misc_feature (203)..(203): n is either C or T
catgaaagtgaaaagtgaaagtgaagtcgctcagtcgtgtccgactcctagcaaccccatggactgcagcccaccaggctcctccatccatgggactctccaggcaagagtactggagtggggtgccattgtcttctctgatgctctctcatacctccaaataattactgctactcattttcagaacctagttacactatctnttcttcataaatgtccttaataacatgctttcaagcactttactatatagcactttgtaacatttaaaattggcaagttatttgaatgattttatgataatgtctagctcctcctgtacagtgaaaactctacaagatcagggagcttgttaatgtttgattcctaatgtatacgtgtgtgtatgtgctaagttgcttcagtggtgcctgattctgtgcgaccccatggactataggctcgtctgtccat

SEQ ID NO: 113 - DNA, 501 bp, Bos taurus
  misc_feature (251)..(251): n is either C or T
ccaagagatgtgatgcgttaacaactgaggggcttctagaacaatcgtagctattaatctttgttgttgttgctgacaaagtgaaaagttcgtccaaagttatagccaattttacaggctggggtttgggtgaggccagcaggacacaagtgattaagggccacagtgataagccccctggtttcaggaggtaactgaggttagaagtgagaaaaccctccaactgctactcatgtaccatcgtccagacncctgtgatgctacctgtccacacaacatgttgtacagtgcctgttggcttactctctctcttttttttgactgcacctcgaggcttgtgggatcttggttccctgaccagggactgaaccctggccaacagcagtgaaagtgctgagtcttaaccactggattgtcagggaagtcccagctcactatctcatttgaccttcatactcatcctgtgttgaagacagagcaagaaccacagtcctattttat

SEQ ID NO: 114 - DNA, 501 bp, Bos taurus
  misc_feature (251)..(251): n is either A or C
agtgagtcaccatttccttctccagggtatcttcccaactcaggggttgaacccacgtctcctgcattaacaggtggattctttactatctgagccaccagagaaaccctagtaagaggctttaaaaaatttttacatctctttggggcagagaaagaatctgtaattacaaggtttctaaagtaagcactctaggaaaagcagggtcaggagtccagagtcagaaagaaatccatctaaagtttagtcangttgagtagaacatgaaggctattttggtcaacatctacttagtggctacccactccaatattctggcctggagaattccatggactatccatcaggccagaaagagtcggacatgactgagcgactttcactttcacttcatgttcaatcacagcaacagtatgtggcctgaacacacttggaaagcaaccagctagaaaaagattgttgaggagccatgcttgtggatgagaacgttccataggtttt

SEQ ID NO: 115 - DNA, 501 bp, Bos taurus
  misc_feature (251)..(251): n is either A or G
caactggagactttgaaaaacttcctgtcagtggctggaatatcataggtctcctgtaggtatttcttgaatgaatgaatgactccagctagctatgactattttaatctgtatttattctttcacagtaaatttgaccataagcatttaatttttaaaatctttttctcaacaatttctcaaattttgtgttaagaacttcctgtaaatacaaaacgcagtaatttaggattatttaaggttacttaaangtagttgatttactttgtgttatgcttctagagaaagcactaaaatttcttggtaaaacttagaaacttcacctaacattttaactattatttttgttttaagacttaacccagttagtcacatgataatgttcttgctttgaaaactttagaaattattgttttgaaagcaggaagtaaatactcatactgagggtagtttattggttctggtacacataatttaaaagctattggtgaaaaggggaag

SEQ ID NO: 116 - DNA, 501 bp, Bos taurus
  misc_feature (251)..(251): n is either C or T
ttgttttagagagtggttgctcatcctggggtcagactccccagtgttcttgggagataattttctcttccctcaggtaggttaagttgggggtaaaaagggcttcttcgaaacaccgtgttaccactgctcaaaaagtcaggaattcctaggaactgcctttcacagcagtttacgaaacacaaattctcaatctctccgttcatgtctctctttctctctcttgcatgtgtatgtgggtgcacacacangctaatttcagagtttatactttttttctgagcaatgtattagcttgacaaccactcaggagtagcagcaaggcacttccggttgtttccacaattaacacccacactcgaagaatgcagctctgcccacagatgaccctggccagagcccatctctgggcaaatgtgtcctctcttgctcacgcccacttgattgtgtttttcctcgcccctgggattcaccctgccttcacgtcctcttcacacctgt

SEQ ID NO: 117 - DNA, 501 bp, Bos taurus
  misc_feature (251)..(251): n is either C or T
aaaagaatttttgccccaagatgaaacaaaaatatactttatttagcaatcttctcaaaatgatgtaagaaaaggattctagttttgattaccaagacatatatggtaagaatctattctttagaattgcttaaattggttaaaaattgaatctttgaaaagagtatagggtgtaattcaaaaaacctgaatacacatagtaactgaatatacataatagatgtaacaattattaaattgttggtttctantatttgtatgtaagtggctgtttctaagatacctgaagtatggttattttcttaactgttttctactctagtaatcttgtttacataatgagtttaaaccgtgttgcaaaataggagtgttgagtgtgtagaaagggtgtgatcacagagtccttcttagggacctcaaaatctcagagaagagtgttcagattccatagctgaagagtcaaacaagctgttgtgcaccaagatgaaaataaactatttc

SEQ ID NO: 118 - DNA, 501 bp, Bos taurus
  misc_feature (251)..(251): n is either A or G
ttgaatttgctgcatgccggcaactaggtacacagccttcttatgttgtatcatgtgttacgagtagcctacacttgatttaaagtatgcgggaagatgtgcatggttttatgctatttcatcgtaggggcctgagcatctgtggatttggtacctgaggagggtcttggaaccagtttctgacagacatgaagggatgactgtgtgttcttattactcacagtactcgtcctacaaagctgctgtgagcnacgcactggcaaacactgacccgtcgctcctcgcggaaatggagttgtgagcctgtgagcctgtgctcatgatactttcatcaacctatcaacacacaaacttgctttgtgtgcattcctgtctaaagacagttcagttcagtcactcagtcgtgtccaactctttgcgaccccatgaaccacagcacgccaggcctccctgtccatcaccaactcccggagttcacccaaactcatgtccatctagtca

SEQ ID NO: 119 - DNA, 501 bp, Bos taurus
  misc_feature (251)..(251): n is either A or G
cctttttgcacaattgcagattgccgcatcccgcaaattgaagatgctgagattcataacaagacatacagacacggagataagctaatcatcacttgtcatgaaggattcaagatccggtaccccgacctatacaatgtggtttcattatgtcgtgatgacggaacgtgggacaatctgcccatctgtcaaggtaggggcaaatggaggctgtggttctcagctctgctttgagactcgactgcagatcnagtgcacgtttctctaataaggtggcagttgtttagtcgctcagtcatgtccaaccctttgcgaccccatggactgtagcctgccaggtttctctgtccatgggattctccaggcaagaatactggagtgggttgccgtgccctcctccaggggatcttcccaacccaaggattgaacccacatctcctgcactggcaggtggattcttgaccactgagccacctgggaagctggcaaatgacgaaggga

SEQ ID NO: 120 - DNA, 501 bp, Bos taurus
  misc_feature (251)..(251): n is either A or G
actgcctatttacactgtgtgggcctttgtaatgactgctatcattacagtgtgctagacgatactctattcttcacatgtttattctttagtcttaaaacaattcaaaagcataggtacagtacaaattaatcatttgaaacctctactataacagacttgagaggccagaaagagaagctttcacaccctatgataataacagagcacaaaaggaagaagaaagacttcctcttctcgctgggcaacanctcagccaatgagaaactgtcagaagtcagccaatgaaaaaccactgtacttagaactctcagttcctcaaatagaacctttgtttatcatagccctcctttcttttccctattatatctactctacttgttgacctggattttcacatggcttatttttgcagaatctgaatcacacttctttgttatcccaaataaacctatctttcactgtagaaataactggcaatctatttatgtaaaatgagca

SEQ ID NO: 121 - DNA, 456 bp, Bos taurus
  misc_feature (206)..(206): n is either A or G
agatgcagtgacccagtaaagttaatgttttgtagaagtaaaggaaagcaatgaacccttgagtatgtgccactcaaggagacaagactgttgactttgcttccatcaagctttgaagcccatgaactggccatgagatagtgcatcttgctctggggcttagaaaagacgtcttcagaacagcgtccactgtgagcattcctgcngacacacagagaggtaaagaaaccactggggtttgttctgccctttgactattgctgaccaattgctgtgggtggtgagttgctatgaattttaattaataccaactgggtcttactatcaaagcactaagcatgccttccccacatgtagacaacctacacacccagtttctcagctgtctctctagttccacctgtgaccaacaagctaagcactttatatgcgtacaggccaccttttatttttatg

SEQ ID NO: 122 - DNA, 41 bp, Bos taurus
  misc_feature (21)..(21): y is either c or t
tcttacacatcaggagatagytccgaggtggatttctacaa

SEQ ID NO: 123 - DNA, 41 bp, Bos taurus; identical to SEQ ID NO: 122
  misc_feature (21)..(21): y is either c or t

SEQ ID NO: 124 - DNA, 41 bp, Bos taurus
  misc_feature (21)..(21): y is either c or t
tcttacacatcaggagatggytccgaggtggatttctacaa

(19) United States
(12) Patent Application Publication
(10) Pub. No.: US 2010/0304353 A1
(43) Pub. Date: Dec. 2, 2010

(54) METHODS OF IMPROVING A GENOMIC MARKER INDEX OF DAIRY ANIMALS AND PRODUCTS

(75) Inventors: Fengxing Du, St. Louis, MI (US); Nicholas J. Nissing, St. Louis, MI (US); Michael D. Grosz, St. Louis, MI (US); Michael M. Lohuis, St. Louis, MI (US)

Correspondence Address: PFIZER INC;Mary J Hosley, 150 EAST 42ND STREET, MS: 150/02/E112, NEW YORK, NY, 10017-5612, US

(73) Assignee: Pfizer Inc

(21) Appl. No.: 12/669,046

(22) PCT Filed: Jul. 15, 2008
(86) PCT No.: PCT/US08/08641

§ 371 Date: Aug. 18, 2010

Related U.S. Application Data
(60) Provisional application No. 60/959,677, filed on Jul. 16, 2007.

(51) Int. Cl. A01N 1/02; C12Q 1/68

(52) U.S. Cl. 435/2; 435/6

(57) ABSTRACT

The present invention provides methods for improving desirable dairy traits through the use of a genomic marker index. Also provided are methods for determining an animal's genotype with respect to multiple markers used in calculation of the genomic marker index. The invention also provides methods for selecting or allocating animals for predetermined uses, for picking potential parent animals for breeding, and for producing improved dairy products.

### PRIORITY CLAIM

This application claims the benefit of U.S. Provisional Application Ser. No. 60/959,677, filed Jul. 16, 2007, which is herein incorporated by reference in its entirety.

### INCORPORATION OF SEQUENCE LISTING

A sequence listing is contained in the file named “Bovine_Product_Claim.st251.txt” which is 96,256 bytes (94.0 kilobytes) (measured in MS-Windows XP) and was created on Jul. 16, 2008 and is located in computer readable form on a compact disc (in accordance with 37 C.F.R. §1.52(e) and 37 C.F.R. §1.1.821), which is enclosed herewith and incorporated herein by reference.

### FIELD OF THE INVENTION

The invention relates to improved genetic profiles of dairy animals, products comprising improved genetic profiles, and methods of producing these products. More specifically, it relates to using genetic markers in methods for improving dairy cattle and dairy products, such as isolated semen, with respect to a variety of performance traits including, but not limited to such traits as, Somatic Cell Score (SCS), Daughter Pregnancy Rate (DPR), Productive Life (PL), Fat Content (FAT), Protein Content (PROT), and Net Merit (NM).

### BACKGROUND OF THE INVENTION

The future viability and competitiveness of the dairy industry depends on continual improvement in milk productivity (e.g. milk production, fat yield, protein yield, fat %, protein % and persistency of lactation), health (e.g. Somatic Cell Count, mastitis incidence), fertility (e.g. pregnancy rate, display of estrus, calving interval and non-return rates in bulls), calving ease (e.g. direct and maternal calving ease), longevity (e.g. productive life), and functional conformation (e.g. udder support, proper foot and leg shape, proper rump angle, etc.). Unfortunately efficiency traits are often unfavorably correlated with fitness traits. Although fitness traits all have some degree of underlying genetic variation in commercial cattle populations, the accuracy of selecting breeding animals with superior genetic merit for many of them is low due to low heritability or the inability to measure the trait cost effectively on the candidate animal. In addition, many productivity and fitness traits can only be measured on females. Thus, the accuracy of conventional selection for these traits is moderate to low and ability to make genetic change through selection is limited, particularly for fitness traits.

Genomics offers the potential for greater improvement in productivity and fitness traits through the discovery of genes, or genetic markers linked to genes, that account for genetic variation and can be used for more direct and accurate selection. Close to 1000 markers with associations with productivity and fitness traits have been reported (see www.bovineqtl.tamu.edu/ for a searchable database of reported QTL), however, the resolution of QTL location is still quite low which makes it difficult to utilize these QTL in marker-assisted selection (MAS) on an industrial scale. Only a few QTL have been fully characterized with a strong putative or well-confirmed causal mutation: DGAT1 on chromosome 14 (Grisard et al., 2002; Winter et al, 2002; Kuhn et al., 2004) GHR on chromosome 20 (Blott et al., 2003), ABCG2 (Cohen-Zinder et al., 2005) or SPP1 on chromosome 6 (Schnabel et al., 2005). However, these discoveries are rare and only explain a small portion of the genetic variance for productivity traits and no genes controlling quantitative fitness traits have been fully characterized. A more successful strategy employs the use of whole-genome high-density scans of the entire bovine genome in which QTL are mapped with sufficient resolution to explain the majority of genetic variation around the traits of interest.

Cattle herds used for milk production around the world originate predominantly from the Holstein or Holstein-Friesian breeds which are known for high levels of production. However, the high production levels in Holsteins have also been linked to greater calving difficulty and reduced levels of fertility. It is unclear whether these unfavorable correlations are due to pleiotropic gene effects or simply due to linked genes. If the latter is true, with marker knowledge, it may be possible to select for favorable recombinants that contain the favorable alleles from several linked genes that are normally at frequencies too low to allow much progress with traditional selection. Since Holstein germplasm has been sold and transported globally for several decades, the Holstein breed has effectively become one large global population held to relatively moderate inbreeding rates. Also, the outbred nature of such a large population selected for several generations has allowed linkage disequilibrium to break down except within relatively short distances (i.e. less than a few centimorgans) (Hayes et al., 2006). Given this pattern of linkage disequilibrium, very dense marker coverage is required to refine QTL locations with sufficient precision to find markers that are in very tight linkage disequilibrium with them. Therefore, markers that are in very tight linkage disequilibrium with the QTL are essential for effective population-wide MAS or whole-genome selection (WGS).

Most traits are quantitative in nature and hence are governed by a large number of QTL of small to moderately sized effects. Therefore, to characterize enough QTL to explain a majority of genetic variation for these traits, a large number of markers need to be evaluated.

Furthermore, a sufficient number of marked QTL must be used in MAS in order to accurately predict the breeding value of an animal without phenotyping records on relatives or the animal itself. The application of such a high-density whole-genome marker map to discover and finely-map QTL explaining variation in productivity and fitness traits is described herein.

The large number of resulting linked markers can be used in several methods of marker selection or marker-assisted selection, including whole-genome selection (WGS) (Meuwissen et al., Genetics 2001) to improve the genetic merit of the population for these traits and create value in the dairy industry.

### SUMMARY OF THE INVENTION

This section provides a non-exhaustive summary of the present invention.

Various embodiments of the invention also provide methods for evaluating an animal's genetic merit at 10 or more positions in the animal's genome and methods of breeding animals using marker assisted selection (MAS). In various aspects of these embodiments the animal's genotype is evaluated at positions within a segment of DNA (an allele) that contains at least one SNP selected from the SNPs described in the Tables and Sequence Listing of the present application.

Other embodiments of the invention provide methods that comprise: a) analyzing the animal's genomic sequence at one or more polymorphisms (where the alleles analyzed each comprise at least one SNP) to determine the animal's genotype at each of those polymorphisms; b) analyzing the genotype determined for each polymorphisms to determine which allele of the SNP is present; c) calculating a genomic marker index for said animal, and d) allocating the animal for use based on its genotype at one or more of the polymorphisms analyzed.

Various aspects of embodiment of the invention provide methods for allocating animals for use based on a genomic marker index using an animal's genotype, at one or more polymorphisms disclosed in the present application. Alternatively, the methods provide for not allocating an animal for a certain use because it has an undesirable genomic marker index which is not associated with desirable phenotypes.

Other embodiments of the invention provide methods for selecting animals for use in breeding to produce progeny. Various aspects of these methods comprise: A) determining the genotype of at least one potential parent animal at one or more locus/loci, where at least one of the loci analyzed contains an allele of a SNP selected from the group of SNPs described in Table 1 and the Sequence Listing. B) Analyzing the determined genotype at one or more positions for at least one animal to determine which of the SNP alleles is present. C) Calculating a genomic marker index for said animal D) Allocating at least one animal for use to produce progeny.

Other embodiments of the invention provide methods for producing offspring animals (progeny animals). Aspects of this embodiment of the invention provide methods that comprise: breeding an animal—where that animal has been selected for breeding by methods described herein—to produce offspring. The offspring may be produced by purely natural methods or through the use of any appropriate technical means, including but not limited to: artificial insemination; embryo transfer (ET), multiple ovulation embryo transfer (MOET), in vitro fertilization (IVF), or any combination thereof.

Other embodiments of the invention provide bovine products with an elevated GMI. In various aspects of these embodiments, these bovine products comprise isolated semen, milk products, or meat products comprising improved genetic content. Preferably, the bovine products comprising improved genetic content further comprise genomic marker indexes of at least about 130, more preferably at least about 132, more preferably at least about 134, more preferably at least about 136, more preferably at least about 138, still more preferably at least about 140.

Other embodiments of the invention provide isolated semen comprising improved genetic content. Preferably, the isolated semen comprising improved genetic content further comprise genomic marker indexes of at least about 130, more preferably at least about 132, more preferably at least about 134, more preferably at least about 136, more preferably at least about 138, still more preferably at least about 140. Various embodiments of the invention also comprise frozen isolated semen, and isolated semen with disproportionate sex determining characteristics, such as for example, greater than naturally occurring frequencies of X chromosomes.

Other embodiments of the invention provide for databases or groups of databases, each database comprising lists of the nucleic acid sequences, which include a plurality of the SNPs described in Table 1 and the Sequence Listing. Preferred aspects of this embodiment of the invention provide for databases comprising the sequences for 30 or more SNPs. Other aspects of these embodiments comprise methods for using a computer algorithm or algorithms that use one or more database(s), each database comprising a plurality of the SNPs described in Table 1 and the Sequence Listing to identify phenotypic traits associated with the inheritance of one or more alleles of the SNPs, and/or using such a database to aid in animal allocation.

### DEFINITIONS

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “allelic association” preferably means: nonrandom deviation of f(AᵢBⱼ) from the product of f(Aᵢ) and f(Bⱼ), which is specifically defined by r²>0.2, where r² is measured from a reasonably large animal sample (e.g., ≧100) and defined as

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

where A₁ represents an allele at one locus, B₁ represents an allele at another locus; f(A₁B₁) denotes frequency of gametes having both A₁ and B₁, f(A₁) is the frequency of A₁, f(B₁) is the frequency of B₁ in a population.

As used herein the terms “allocating animals for use” and “allocation for use” preferably mean deciding how an animal will be used within a herd or that it will be removed from the herd to achieve desired herd management goals. For example, an animal might be allocated for use as a breeding animal or allocated for sale as a non-breeding animal (e.g. allocated to animals intended to be sold for meat). In certain aspects of the invention, animals may be allocated for use in sub-groups within the breeding programs that have very specific goals (e.g. productivity or fitness). Accordingly, even within the group of animals allocated for breeding purposes, there may be more specific allocation for use to achieve more specific and/or specialized breeding goals.

As used herein, “semen with disproportionate sex determining characteristics” refers to semen that has been modified or otherwise processed to increase the statistical probability of producing offspring of a pre-determined gender when that semen is used to fertilize an oocyte.

As used herein, the term “bovine product” refers to products derived from, produced by, or comprising bovine cells, including but not limited to milk, cheese, butter, yoghurt, ice cream, meat, and leather; as well as biological material used in production of bovine products including for example, isolated semen, embryos, or other reproductive materials.

As used herein, the term “isolated semen” refers to biological material comprising a plurality of sperm/semen which is physically separated from the originating animal, typically as part of a process employing human and/or mechanical intervention. Examples of isolated semen may include but are not limited to straws of semen, frozen straws of semen, and semen suitable for use in IVF procedures.

As used herein, the term “genomic marker index” (GMI) is a numerical representation of the value of genetic content based on the allelic profile of a plurality of genomic markers. Methods to determine specific genomic marker indexes are specified below.

As used herein the terms “animal” or “animals” preferably refer to dairy cattle.

As used herein “fitness” preferably refers to traits that include, but are not limited to: pregnancy rate (PR), daughter pregnancy rate (DPR), productive life (PL), somatic cell count (SCC) and somatic cell score (SCS).

As used herein, PR and DPR refer to the percentage of non-pregnant animals that become pregnant during each 21-day period.

As used herein, PL is calculated as months in each lactation, summed across all lactations until removal of the cow from the herd (by culling or death).

As used herein, somatic cell score can be calculated using the following relationship: SCS=log₂(SCC/100,000)+3, where SCC is somatic cells per milliliter of milk.

As used herein the term “growth” refers to the measurement of various parameters associated with an increase in an animal's size and/or weight.

As used herein the term “linkage disequilibrium” preferably means allelic association wherein A₁ and B₁ (as used in the above definition of allelic association) are present on the same chromosome.

As used herein the term “marker-assisted selection (MAS) preferably refers to the selection of animals on the basis of marker information in possible combination with pedigree and phenotypic data.

As used herein the term “natural breeding” preferably refers to mating animals without human intervention in the fertilization process. That is, without the use of mechanical or technical methods such as artificial insemination or embryo transfer. The term does not refer to selection of the parent animals.

As used herein the term “net merit” preferably refers to a composite index that includes several commonly measured traits weighted according to relative economic value in a typical production setting and expressed as lifetime economic worth per cow relative to an industry base. Examples of a net merit indexes include, but are not limited to, $NM or TPI in the USA, LPI in Canada, etc (formulae for calculating these indices are well known in the art (e.g. $NM can be found on the USDA/AIPL website: www.aipl.arsusda.gov/reference.htm)

As used herein, the term “milk production” preferably refers to phenotypic traits related to the productivity of a dairy animal including milk fluid volume, fat percent, protein percent, fat yield, and protein yield.

As used herein the term “predicted value” preferably refers to an estimate of an animal's breeding value or transmitting ability based on its genotype and pedigree.

As used herein “productivity” and “production” preferably refers to yield traits that include, but are not limited to: total milk yield, milk fat percentage, milk fat yield, milk protein percentage, milk protein yield, total lifetime production, milking speed and lactation persistency.

As used herein the term “quantitative trait” is used to denote a trait that is controlled by multiple (two or more, and often many) genes each of which contributes small to moderate effect on the trait. The observations on quantitative traits often follow a normal distribution.

As used herein the term “quantitative trait locus (QTL)” is used to describe a locus that contains polymorphism that has an effect on a quantitative trait.

As used herein the term “reproductive material” includes, but is not limited to semen, spermatozoa, ova, embryos, and zygote(s).

As used herein the term “single nucleotide polymorphism” or “SNP” refer to a location in an animal's genome that is polymorphic within the population. That is, within the population some individual animals have one type of base at that position, while others have a different base. For example, a SNP might refer to a location in the genome where some animals have a “G” in their DNA sequence, while others have a “T”.

As used herein the term “whole-genome analysis” preferably refers to the process of QTL mapping of the entire genome at high marker density (i.e. at least about one marker per cM) and detection of markers that are in population-wide linkage disequilibrium with QTL.

As used herein the term “whole-genome selection (WGS)” preferably refers to the process of marker-assisted selection (MAS) on a genome-wide basis in which markers spanning the entire genome at moderate to high density (e.g. at least about one marker per 1-5 cM), or at moderate to high density in QTL regions, or directly neighboring or flanking QTL that explain a significant portion of the genetic variation controlling one or more traits.

### BRIEF DESCRIPTION OF THE FIGURE

FIG. 1 depicts the ranges of calculated GMI values for several species of bovine. In each case, the vertical bar represents the range of values calculated, with the horizontal mark indicating the average GMI of the population tested.

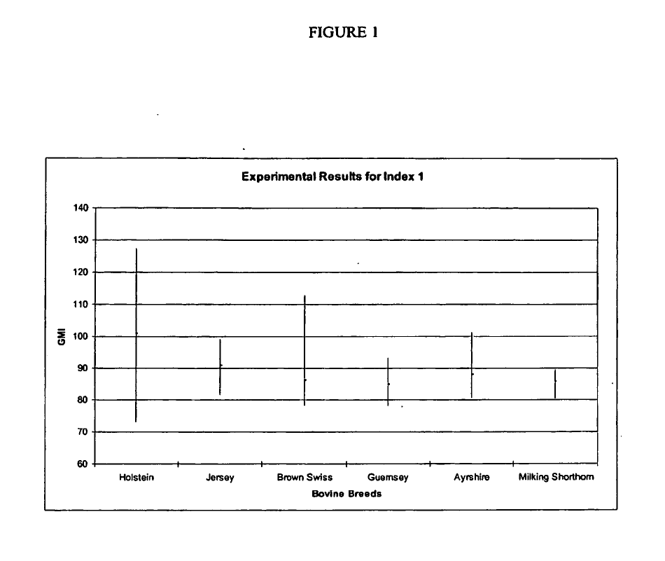

### ILLUSTRATIVE EMBODIMENTS OF THE INVENTION

Various embodiments of the present invention provide methods for evaluating the genomic marker index of a dairy animal or bovine product. In preferred embodiments of the invention, the animal's genotype is evaluated at 10 or more positions (i.e. with respect to 10 or more genetic markers). Aspects of these embodiments of the invention provide methods that comprise determining the animal's genomic sequence at 10 or more locations (loci) that contain single nucleotide polymorphisms (SNPs). Specifically, the invention provides methods for evaluating an animal's genotype by determining which of two or more alleles for the SNP are present for each of 10 or more SNPs selected from the group consisting of the SNPs described in Table 1 and the Sequence Listing.

In preferred aspects of these embodiments the animal's genotype is evaluated to determine which allele is present for SNPs selected from the group of SNPs described in Table 1 and the Sequence Listing.

In other aspects of this embodiment, the animal's genotype is analyzed with respect to SNPs that have been shown to be associated with one or more traits (see Table 1) and are used to calculate a genomic marker index. For example, embodiments of the invention provides a method for genotyping 10 or more, 25 or more, 50 or more, 100 or more, 200 or more, or 500 or more, or 1000 or more SNPs that have been determined to be significantly associated with one or more of these traits. These SNPs are preferably selected from the group consisting of the SNPs described in Table 1 and the Sequence Listing.

Aspects of the present invention also provides for both whole-genome analysis and whole genome-selection (WGS) (i.e. marker-assisted selection (MAS) on a genome-wide basis). Moreover the invention provides that of the markers used to carry out the whole-genome analysis or WGS, 10 or more, 25, or more, 50 or more, 100 or more are selected from the group consisting of the markers described in Table 1 and the Sequence Listing.

In any embodiment of the invention the genomic sequence at the SNP locus may be determined by any means compatible with the present invention. Suitable means are well known to those skilled in the art and include, but are not limited to direct sequencing, sequencing by synthesis, primer extension, Matrix Assisted Laser Desorption/Ionization-Time Of Flight (MALDI-TOF) mass spectrometry, polymerase chain reaction-restriction fragment length polymorphism, microarray/multiplex array systems (e.g. those available from Illumina Inc., San Diego, Calif. or Affymetrix, Santa Clara, Calif.), and allele-specific hybridization.

Other embodiments of the invention provide methods for allocating animals for subsequent use (e.g. to be used as sires or dams or to be sold for meat or dairy purposes) according to their predicted value for productivity or fitness. Various aspects of this embodiment of the invention comprise determining at least one animal's genotype for at least one SNP selected from the group of SNPs consisting of the SNPs described in Table 1 and the sequence listing, (methods for determining animals' genotypes for one or more SNPs are described supra). Thus, the animal's allocation for use may be determined based on its genotype and resulting genomic marker index.

The instant invention also provides embodiments where analysis of the genotypes of the SNPs described in Table 1 and the Sequence Listing is the only analysis done. Other embodiments provide methods where analysis of the SNPs disclosed herein is combined with any other desired type of genomic or phenotypic analysis (e.g. analysis of any genetic markers beyond those disclosed in the instant invention).

According to various aspects of these embodiments of the invention, once the animal's genetic sequence for the selected SNP(s) have been determined, this information is evaluated to determine which allele of the SNP is present for selected SNPs. Preferably the animal's allelic complement for all of the determined SNPs is evaluated. Next, a genomic marker index is calculated based on specific methods described below. Finally, the animal is allocated for use based on its genotype for one or more of the SNP positions evaluated. Preferably, the allocation is made taking into account the animal's genomic marker index.

The allocation may be made based on any suitable criteria. For any genomic marker index, a determination may be made as to whether an animal's GMI exceeds target values. This determination will often depend on breeding or herd management goals. Additionally, other embodiments of the invention provide methods where combinations of two or more criteria are used. Such combinations of criteria include but are not limited to, two or more criterion selected from the group consisting of: phenotypic data, pedigree information, breed information, the animal's GMI, and GMI information from siblings, progeny, and/or parents.

Determination of which alleles are associated with desirable phenotypic characteristics can be made by any suitable means. Methods for determining these associations are well known in the art; moreover, aspects of the use of these methods are generally described in the EXAMPLES, below.

According to various aspects of this embodiment of the invention allocation for use of the animal may entail either positive selection for the animals having the desired genomic marker index (e.g. the animals with the desired genotypes are selected), negative selection of animals having an undesirable genomic marker index (e.g. animals with a GMI lower than a pre-determined threshold), or any combination of these methods.

According to preferred aspects of this embodiment of the invention, animals or bovine products identified as having a genomic marker index above a minimum threshold are allocated to a use consistent with animals having higher economic value. Alternatively, animals or bovine products that have a GMI lower than the minimum threshold are not allocated for the same use as those with a higher GMI.

Other embodiments of the invention provide methods for selecting potential parent animals (i.e., allocation for breeding) to improve fitness and/or productivity in potential offspring. Various aspects of this embodiment of the invention comprise determining at least one animal's GMI using SNPs selected from the group of SNPs consisting of the SNPs described in Table 1 and the Sequence Listing. Furthermore, determination of whether and how an animal will be used as a potential parent animal may be based on its genomic marker index, pedigree information, breed information, phenotypic information, progeny information, or any combinations thereof.

Moreover, as with other types of allocation for use, various aspects of these embodiments of the invention provide methods where the only analysis done is to calculate the genomic marker index. Other aspects of these embodiments provide methods where analysis of the genomic marker index disclosed herein is combined with any other desired genomic or phenotypic analysis (e.g. analysis of any genetic markers beyond those disclosed in the instant invention).

According to various aspects of these embodiments of the invention, once the animal's genetic sequence at the site of the selected SNP(s) have been determined, this information is evaluated to determine which allele of the SNP is present for at least one of the selected SNPs. Preferably the animal's allelic complement for all of the sequenced SNPs is evaluated. Additionally, the animal's allelic complement is analyzed and evaluated to calculate the genomic marker index and thereby predict the animal's progeny's genetic merit or phenotypic value. Finally, the animal is allocated for use based on its genomic marker index, either alone or in combination with one or more additional criterion/criteria.

Other embodiments of the instant invention provide methods for producing progeny animals. According to various aspects of this embodiment of the invention, the animals used to produce the progeny are those that have been allocated for breeding according to any of the embodiments of the current invention. Those using the animals to produce progeny may perform the necessary analysis or, alternatively, those producing the progeny may obtain animals that have been analyzed by another. The progeny may be produced by any appropriate means, including, but not limited to using: (i) natural breeding, (ii) artificial insemination, (iii) in vitro fertilization (IVF) or (iv) collecting semen/spermatozoa and/or at least one ovum from the animal and contacting it, respectively with ova/ovum or semen/spermatozoa from a second animal to produce a conceptus by any means.

According to other aspects of the invention, the progeny are produced through a process comprising the use of standard artificial insemination (AI), in vitro fertilization, multiple ovulation embryo transfer (MOET), or any combination thereof.

Other embodiments of the invention provide for bovine products having a GMI greater than a pre-determined threshold. Preferably, these bovine products have a GMI of at least about 130, more preferably at least about 132, more preferably at least about 134, more preferably at least about 136, more preferably at least about 138, still more preferably at least about 140. In various aspects of these embodiments, these bovine products include but are not limited to isolated semen, reproductive materials, dairy products, meat products, spermatozoa, ovum, zygotes, blood, tissue, serum, and the like.

Other embodiments of the invention provide for bovine animals having a GMI greater than a pre-determined threshold. Preferably, these bovine products have a GMI of at least about 130, more preferably at least about 132, more preferably at least about 134, more preferably at least about 136, more preferably at least about 138, still more preferably at least about 140.

Other embodiments of the invention provide for methods that comprise allocating an animal for breeding purposes and collecting/isolating genetic material from that animal: wherein genetic material includes but is not limited to: semen, spermatozoa, ovum, zygotes, blood, tissue, serum, DNA, and RNA.

It is understood that most efficient and effective use of the methods and information provided by the instant invention employ computer programs and/or electronically accessible databases that comprise all or a portion of the sequences disclosed in the instant application. Accordingly, the various embodiments of the instant invention provide for databases comprising all or a portion of the sequences corresponding to at least 10 SNPs described in Table 1 and the Sequence Listing. In preferred aspect of these embodiments the databases comprise sequences for 25 or more, 50 or more, 100 or more, or substantially all of the SNPs described in Table 1 and the Sequence Listing.

It is further understood that efficient analysis and use of the methods and information provided by the instant invention will employ the use of automated genotyping. Any suitable method known in the art may be used to perform such genotyping, including, but not limited to the use of micro-arrays.

Other embodiments of the invention provide methods wherein one or more of the SNP sequence databases described herein are accessed by one or more computer-executable programs. Such methods include, but are not limited to, use of the databases by programs to analyze for an association between the SNP and a phenotypic trait, or other user-defined trait (e.g. traits measured using one or more metrics such as gene expression levels, protein expression levels, or chemical profiles), calculation of a genomic marker index, and programs used to allocate animals for breeding or market.

Other embodiments of the invention provide methods comprising collecting genetic material and calculating a genomic marker index from an animal that has been allocated for breeding. Wherein the animal has been allocated for breeding by any of the methods disclosed as part of the instant invention.

Other embodiments of the invention provide for diagnostic kits or other diagnostic devices for determining which allele of one or more SNP(s) is/are present in a sample; wherein the SNP(s) are selected from the group of SNPs consisting of the SNPs described in Table 1 and the sequence listing. In various aspects of this embodiment of the invention, the kit or device provides reagents/instruments to facilitate a determination as to whether nucleic acid corresponding to the SNP is present. Such kit/or device may further facilitate a determination as to which allele of the SNP is present. In certain aspects of this embodiment of the invention the kit or device comprises at least one nucleic acid oligonucleotide suitable for DNA amplification (e.g. through polymerase chain reaction). In other aspects of the invention the kit or device comprises a purified nucleic acid fragment capable of specifically hybridizing, under stringent conditions, with at least one allele of at least ten of the SNPs described in Table 1 and the Sequence listing.

In particularly preferred aspects of this embodiment of the invention the kit or device comprises at least one nucleic acid array (e.g. DNA micro-arrays) capable of determining which allele of one or more of the SNPs are present in a sample; where the SNPs are selected from the group of SNPs consisting of the SNPs described in Table 1 and the Sequence Listing. Preferred aspects of this embodiment of the invention provide DNA micro-arrays capable of simultaneously determining which allele is present in a sample for 10 or more SNPs. Preferably, the DNA micro-array is capable of determining which SNP allele is present in a sample for 25 or more, 50 or more, 100 or more SNPs. Methods for making such arrays are known to those skilled in the art and such arrays are commercially available (e.g. from Affymetrix, Santa Clara, Calif.).

Genetic markers that are in allelic association with any of the SNPs described in the Tables may be identified by any suitable means known to those skilled in the art. For example, a genomic library may be screened using a probe specific for any of the sequences of the SNPs described in the Tables. In this way clones comprising at least a portion of that sequence can be identified and then up to 300 kilobases of 3′ and/or 5′ flanking chromosomal sequence can be determined. Preferably up to about 70 kilobases of 3′ and/or 5′ flanking chromosomal sequences are evaluated. By this means, genetic markers in allelic association with the SNPs described in the Tables will be identified. These alternative markers in allelic association may be used to select animals in place of the markers described in Table 1 and the sequence listing.

In preferred embodiments of the invention, a genomic marker index (GMI) is calculated based on genotypic information acquired from a dairy animal or bovine product. The genomic marker index has been created based on the whole genome genetic analysis described above. The index was created using the trait association, effect estimates, and expected values of the underlying markers.

The following equation is used to calculate the genomic marker index, in conjunction with Table 1. Specifically, the variables in the equation are defined by the weighted coefficients listed in the table for each respective marker.

The first step is to genotype all of 121 markers that are described in Table 1 for an animal. With the resulting genotype data, the i^{th} genomic marker index of the animal (i.e., the k^{th} animal) can be determined using following equation:

$${GMI}_{ik} = {\sum }_{j = 1}^{121} {W}_{ij} ( {G}_{jk} ) \qquad [ Equation 2 ]$$

where Gⱼₖ is the genotype of j^{th} marker of bull k; Wᵢⱼ(Gⱼₖ) is the weight of genotype Gⱼₖ at the j^{th} marker for index i. The values listed in Table 1 correspond to the weighting for a single strand of DNA. Therefore, each genotype will have two values for each SNP, one for each allele. A homozygous value will be two times the weighting for the respective allele, while a heterozygous value will be the sum of each allele weighting. For example, a sample which is homozygous for the G allele at SNP1 (e.g., GG) would include a weighting equal to 2× the weighting listed for the G allele in table 1. A sample which is heterozygous for the SNP1 (e.g., GA) would include a weighting equal to the sum of the weighting for the G allele and the weighting for the A allele.

For example, the GMI for index 1 of a bull would be calculated as follows:

Genotype of SNP 1=GG, weighting=0.45621+0.45621=0.91242

Genotype of SNP 2=GA, weighting=0.174516+0.480119=0.657895

Genotype of SNP 3=TT, weighting=(−0.13095)+(−0.13095)=−0.26191
. . .

Genotype of SNP 121=AG, weighting=0.642706+0.071233=0.713936

Therefore, GMI₁=0.91242+0.657895+(−0.26191)+0.713936

Other embodiments of the invention provide isolated semen comprising improved genetic content. Preferably, the isolated semen comprising improved genetic content further comprise genomic marker indexes of at least about at least about 130, more preferably at least about 132, more preferably at least about 134, more preferably at least about 136, more preferably at least about 138, still more preferably at least about 140. Various embodiments of the invention also comprise frozen isolated semen, and isolated semen with disproportionate sex determining characteristics, such as for example, greater than naturally occurring frequencies of X chromosomes.

When determining the GMI of sperm, the GMI is determined based on all alleles present in the source animal for each SNP, including those homozygous for each allele and heterozygous for combinations of alleles. Because each individual sperm and unfertilized egg contains only a haploid genome (as opposed to a diploid genome), the GMI calculations provided herein are only applicable in those instances where a sufficient number of haploid cells are present to determine the diploid genotype of the animal from which the cells were derived (ie. greater than about 50 individual cells).

When determining the GMI of other bovine products, at least one DNA sample must be retrieved from the product. For example, when testing milk, DNA may be retrieved from the leucocytes cells contained therein. When testing bovine meat products, DNA can be extracted from the muscle fibers. Preferably when evaluating the GMI of bovine products, DNA from at least about 50 individual cells are used to determine the GMI. However, recent advances in the field of DNA extraction and replication allow for determining genetic content from a sample as small as one cell (Zhang, 2006).

Methods of collecting, storing, freezing, and using isolated semen are well known in the art. Any suitable techniques can be utilized in conjunction with the genomic marker index described herein. Furthermore, techniques for altering sex determining characteristics such as the frequency of X chromosomes in the sperm suspension are also known. A variety of methods for altering sex determining characteristics are known in the art, including for example, cell cytometry, photodamage, and microfluidics. The following references related to methods of collecting, storing, freezing, and altering sex-determining characteristics of sperm suspensions are hereby incorporated by reference: U.S. Pat. No. 5,135,759, U.S. Pat. No. 5,985,216, U.S. Pat. No. 6,071,689, U.S. Pat. No. 6,149,867, U.S. Pat. No. 6,263,745, U.S. Pat. No. 6,357,307, U.S. Pat. No. 6,372,422, U.S. Pat. No. 6,524,860, U.S. Pat. No. 6,604,435, U.S. Pat. No. 6,617,107, U.S. Pat. No. 6,746,873, U.S. Pat. No. 6,782,768, U.S. Pat. No. 6,819,411, U.S. Pat. No. 7,094,527, U.S. Pat. No. 7,169,548, US2002005076A1, US2002096123A1, US2002119558A1, US2002129669A1, US2003157475A1, US2004031071A1, US2004049801A1, US2004050186A1, US2004053243A1, US2004055030A1, US2005003472A1, US2005112541A1, US2005130115A1, US2005214733A1, US2005244805A1, US2005282245A1, US2006067916A1, US2006118167A1, US2006121440A1, US2006141628A1, US2006170912A1, US2006172315A1, US2006229367A1, US2006263829A1, US2006281176A1, US2007026378A1, US2007026379A1, US2007042342A1.

### EXAMPLES

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

### Example 1

### Determining Associations Between Genetic Markers and Phenotypic Traits or Indexes

Simultaneous discovery and fine-mapping on a genome-wide basis of genes underlying quantitative traits (Quantitative Trait Loci: QTL) requires genetic markers densely covering the entire genome. As described in this example, a whole-genome, dense-coverage marker map was constructed from microsatellite and single nucleotide polymorphism (SNP) markers with previous estimates of location in the bovine genome, and from SNP markers with putative locations in the bovine genome based on homology with human sequence and the human/cow comparative map. A new linkage-mapping software package was developed, as an extension of the CRIMAP software (Green et al., Washington University School of Medicine, St. Louis, 1990), to allow more efficient mapping of densely-spaced markers genome-wide in a pedigreed livestock population (Liu and Grosz Abstract C014; Grapes et al. Abstract W244; 2006 Proceedings of the XIV Plant and Animal Genome Conference, www.intl-pag.org). The new linkage mapping tools build on the basic mapping principles programmed in CRIMAP to improve efficiency through partitioning of large pedigrees, automation of chromosomal assignment and two-point linkage analysis, and merging of sub-maps into complete chromosomes. The resulting whole-genome discovery map (WGDM) included 6,966 markers and a map length of 3,290 cM for an average map density of 2.18 markers/cM. The average gap between markers was 0.47 cM and the largest gap was 7.8 cM. This map provided the basis for whole-genome analysis and fine-mapping of QTL contributing to variation in productivity and fitness in dairy cattle.

### Discovery and Mapping Populations

Systems for discovery and mapping populations can take many forms. The most effective strategies for determining population-wide marker/QTL associations include a large and genetically diverse sample of individuals with phenotypic measurements of interest collected in a design that allows accounting for non-genetic effects and includes information regarding the pedigree of the individuals measured. In the present example, an outbred population following the grand-daughter design (Weller et al., 1990) was used to discover and map QTL: the population, from the Holstein breed, had 529 sires each with an average of 6.1 genotyped sons, and each son has an average of 4216 daughters with milk data. DNA samples were collected from approximately 3,200 Holstein bulls and about 350 bulls from other dairy breeds; representing multiple sire and grandsire families.

### Phenotypic Analyses

Dairy traits under evaluation include traditional traits such as milk yield (“MILK”) (pounds), fat yield (“FAT”) (pounds), fat percentage (“FATPCT”) (percent), productive life (“PL”) (months), somatic cell score (“SCS”) (Log), daughter pregnancy rate (“DPR”) (percent), protein yield (“PROT”) (pounds), protein percentage (“PROTPCT”) (percent), and net merit (“NM”) (dollar), and combinations of multiple traits, such as for example a GMI. These traits are sex-limited, as no individual phenotypes can be measured on male animals. Instead, genetic merits of these traits defined as PTA (predicted transmitting ability) were estimated using phenotypes of all relatives. Most dairy bulls were progeny tested with a reasonably larger number of daughters (e.g., >50), and their PTA estimation is generally more or considerably more accurate than individual cow phenotype data. The genetic evaluation for traditional dairy traits of the US Holstein population is performed quarterly by USDA. Detailed descriptions of traits, genetic evaluation procedures, and genetic parameters used in the evaluation can be found at the USDA AIPL web site (www.aipl.arsusda.gov). It is meaningful to note that the dairy traits evaluated in this example are not independent: FAT and PROT are composite traits of MILK and FATPCT, and MILK and PROTPCT, respectively. NM is an index trait calculated based on protein yield, fat yield, production life, somatic cell score, daughter pregnancy, calving difficulty, and several type traits. Protein yield and fat yield together account for >50% of NM, and the value of milk yield, fat content, and protein content is accounted for via protein yield and fat yield.

PTA data of all bulls with progeny testing data were downloaded from the USDA evaluation published at the AIPL site in February 2007. The PTA data were analyzed using the following two models:

yᵢⱼ=sᵢ+PTAdᵢⱼ [Equation 4]

yᵢ=μ+β₁(SPTA)ᵢ+PTAdᵢ [Equation 5]

where yᵢ (yᵢⱼ) is the PTA of the i^{th} bull (PTA of the j^{th} son of the i^{th} sire); sᵢ is the effect of the i^{th} sire; (SPTA)ᵢ, is the sire's PTA of the i^{th} bull of the whole sample; μ is the population mean; PTAdᵢ (PTAdᵢⱼ) is the residual bull PTA.

Equation 4 is referred to as the sire model, in which sires were fitted as fixed factors. Among all USA Holstein progeny tested bulls, a considerably large number of sires only have a very small number of progeny tested sons (e.g., some have one son), and it is clearly undesirable to fit sires as fixed factors in these cases. It is well known the USA Holstein herds have been making steady and rapid genetic progress in traditional dairy traits in the last several decades, implying that the sire's effect can be partially accounted for by fitting the birth year of a bull. For sires with <10 progeny tested sons, sires were replaced with son's birth year in Equation 4. Equation 5 is referred to as the SPTA model, in which sire's PTA are fitted as a covariate. Residual PTA (PTAdᵢ or PTAdᵢⱼ) were estimated using linear regression.

### Example 2

### Use of Single Nucleotide Polymorphisms to Improve Offspring Traits

To improve the average genetic merit of a population for a chosen trait, one or more of the markers with significant association to that trait can be used in selection of breeding animals. In the case of each discovered locus, use of animals possessing a marker allele (or a haplotype of multiple marker alleles) in population-wide Linkage Disequilibrium (LD) with a favorable QTL allele will increase the breeding value of animals used in breeding, increase the frequency of that QTL allele in the population over time and thereby increase the average genetic merit of the population for that trait. This increased genetic merit can be disseminated to commercial populations for full realization of value.

Furthermore, multiple markers can be used simultaneously, such as for example, when improving offspring traits using a GMI. In this case, a plurality of markers are measured and weighted according to the value of the associated traits and the estimated effect of the marker on the trait. The calculation of a GMI allows inclusion of multiple traits and markers simultaneously with their associated values, thereby optimizing multiple parameters of the selection process.

For example, a progeny-testing scheme could greatly improve its rate of genetic progress or graduation success rate via the use of markers for screening juvenile bulls. Typically, a progeny testing program would use pedigree information and performance of relatives to select juvenile bulls as candidates for entry into the program with an accuracy of approximately 0.5. However, by adding marker information, young bulls could be screened and selected with much higher accuracy. In this example, DNA samples from potential bull mothers and their male offspring could be screened with a genome-wide set of markers in linkage disequilibrium with QTL, and the bull-mother candidates with the best marker profile could be contracted for matings to specific bulls.

Alternatively, a set of markers associated with phenotypic traits could be used to create a GMI, and the bull-mother candidates with GMIs above pre-determined thresholds could be contracted for matings to specific bulls. Furthermore, combinations of GMI, associated markers, phenotypic data, pedigree information, and other historical performance parameters can be used simultaneously.

If superovulation and embryo transfer (ET) is employed, a set of 5-10 offspring could be produced per bull mother per flush procedure. Then the marker set could again be used to select the best male offspring as a candidate for the progeny test program. If genome-wide markers are used, it was estimated that accuracies of marker selection could reach as high as 0.85 (Meuwissen et al., 2001). This additional accuracy could be used to greatly improve the genetic merit of candidates entering the progeny test program and thereby increasing the probability of successfully graduating a marketable progeny-tested bulls. This information could also be used to reduce program costs by decreasing the number of juvenile bull candidates tested while maintaining the same number of successful graduates. In the extreme, very accurate Genomic Marker Indexes (GMIs) could be used to directly market semen from juvenile sires without the need of progeny-testing at all. Due to the fact that juveniles could now be marketed starting at puberty instead of 4.5 to 5 years, generation interval could be reduced by more than half and rates of gain could increase as much as 68.3% (Schrooten et al., 2004). With the elimination of the need for progeny testing, the cost of genetic improvement for the artificial insemination industry would be vastly improved (Schaeffer, 2006).

In an alternate example, a centralized or dispersed genetic nucleus (GN) population of cattle could be maintained to produce juvenile bulls for use in progeny testing or direct sale on the basis of GMIs. A GN herd of 1000 cows could be expected to produce roughly 3000 offspring per year, assuming the top 10-15% of females were used as ET donors in a multiple-ovulation and embryo-transfer (MOET) scheme. However, markers could change the effectiveness of MOET schemes and in vitro embryo production. Previously, MOET nucleus schemes have proven to be promising from the standpoint of extra genetic gain, but the costs of operating a nucleus herd together with the limited information on juvenile animals has limited widespread adoption. However, with marker information and/or GMIs, juveniles can be selected much more accurately than before resulting in greatly reduced generation intervals and boosted rates of genetic response. This is especially true in MOET nucleus herd schemes because, previously, breeding values of full-sibs would be identical, but with marker information the best full-sib can be identified early in life. The marker information and/or GMI would also help limit inbreeding because less selection pressure would be placed on pedigree information and more on individual marker information. An early study (Meuwissen and van Arendonk, 1992) found advantages of up to 26% additional genetic gain when markers were employed in nucleus herd scenarios; whereas, the benefit in regular progeny testing was much less.

Together with MAS, female selection could also become an important source of genetic improvement particularly if markers explain substantial amounts of genetic variation. Further efficiencies could be gained by marker testing of embryos prior to implantation (Bredbacka, 2001). This would allow considerable selection to occur on embryos such that embryos with inferior marker profiles could be discarded prior to implantation and recipient costs. This would again increase the cost effectiveness of nucleus herds because embryo pre-selection would allow equal progress to be made with a smaller nucleus herd. Alternatively, this presents further opportunities for pre-selection prior to bulls entering progeny test and rates of genetic response predicted to be up to 31% faster than conventional progeny testing (Schrooten et al., 2004).

The first step in using a GMI for estimation of breeding value and selection in the GN is collection of DNA from all offspring that will be candidates for selection as breeders in the GN or as breeders in other commercial populations (in the present example, the 3,000 offspring produced in the GN each year). One method is to capture shortly after birth a small bit of ear tissue, hair sample, or blood from each calf into a labeled (bar-coded) tube. The DNA extracted from this tissue can be used to assay a large number of SNP markers. Then the animal's GMI can be calculated and the results used in selection decisions before the animal reaches breeding age.

One method for incorporating into selection decisions the markers (or marker haplotypes) determined to be in population-wide LD with valuable QTL alleles (see Example 1) is based on classical quantitative genetics and selection index theory (Falconer and Mackay, 1996; Dekkers and Chakraborty, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker:

α₁=q[a+d(q−p)] [Equation 6]

α₂=−p[a+d(q−p)] [Equation 7]

α=a+d(q−p) [Equation 8]

g_{A1A1}=2(α₁) [Equation 9]

g_{A1A2}=(α₁)+(α₂) [Equation 10]

g_{A2A2}=2(α₂) [Equation 11]

where αₗ and α₂ are the average effects of alleles 1 and 2, respectively; α is the average effect of allele substitution; p and q are the frequencies in the population of alleles 1 and 2, respectively; a and d are additive and dominance effects, respectively; g_{A1A1}, g_{A1A2} and g_{A2A2} are the (marker) breeding values for animals with marker genotypes A1A1, A1A2 and A2A2, respectively. The total trait breeding value for an animal is the sum of breeding values for each marker (or haplotype) considered and the residual polygenic breeding value:

EBVᵢⱼ=Σĝⱼ+Ûᵢ [Equation 12]

where EBVᵢⱼ is the Estimated Trait Breeding Value for the i^{th} animal, Σ ĝⱼ is the marker breeding value summed from j=1 to n where n is the total number of markers (haplotypes) under consideration, and Ûᵢ is the polygenic breeding value for the i^{th} animal after fitting the marker genotype(s).

These methods can readily be extended to estimate breeding values for selection candidates for multiple traits including GMIs. The breeding value for each trait including information from multiple markers (haplotypes), are all within the context of selection index theory and specific breeding objectives that set the relative importance of each trait. Other methods also exist for optimizing marker information in estimation of breeding values for multiple traits, including random models that account for recombination between markers and QTL (e.g., Fernando and Grossman, 1989), and the potential inclusion of all discovered marker information in whole-genome selection (Meuwissen et al., Genetics 2001). Through any of these methods, the markers reported herein that have been determined to be in population-wide LD with valuable QTL alleles may be used to provide greater accuracy of selection, greater rate of genetic improvement, and greater value accumulation in the dairy industry.

### Example 3

### Identification of SNPs

A nucleic acid sequence contains a SNP of the present invention if it comprises at least 20 consecutive nucleotides that include and/or are adjacent to a polymorphism described in Table 1 and the Sequence Listing. Alternatively, a SNP may be identified by a shorter stretch of consecutive nucleotides which include or are adjacent to a polymorphism which is described in Table 1 and the Sequence Listing in instances where the shorter sequence of consecutive nucleotides is unique in the bovine genome. A SNP site is usually characterized by the consensus sequence in which the polymorphic site is contained, the position of the polymorphic site, and the various alleles at the polymorphic site. “Consensus sequence” means DNA sequence constructed as the consensus at each nucleotide position of a cluster of aligned sequences.

Such SNP have a nucleic acid sequence having at least 90% sequence identity, more preferably at least 95% or even more preferably for some alleles at least 98% and in many cases at least 99% sequence identity, to the sequence of the same number of nucleotides in either strand of a segment of animal DNA which includes or is adjacent to the polymorphism. The nucleotide sequence of one strand of such a segment of animal DNA may be found in a sequence in the group consisting of SEQ ID NO:1 through SEQ ID NO:124. It is understood by the very nature of polymorphisms that for at least some alleles there will be no identity at the polymorphic site itself. Thus, sequence identity can be determined for sequence that is exclusive of the polymorphism sequence. The polymorphisms in each locus are described in the sequence listing.

Shown below are examples of public bovine SNPs that match each other:

SNP ss38333809 was determined to be the same as ss38333810 because 41 bases (with the polymorphic site at the middle) from each sequence match one another perfectly (match length=41, identity=100%).

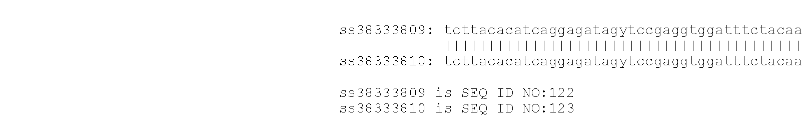

SNP ss38333809 was determined to be the same as ss38334335 because 41 bases (with the polymorphic site at the middle) from each sequence match one another at all bases except for one base (match length=41, identity=97%).

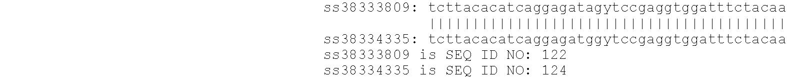

### Example 4

### Quantification of and Genetic Evaluation for Production Traits

Quantifying production traits can be accomplished by measuring milk of a cow and milk composition at each milking, or in certain time intervals only. In the USDA yield evaluation the milk production data are collected by Dairy Herd Improvement Associations (DHIA) using ICAR approved methods. Genetic evaluation includes all cows with the known sire and the first calving in 1960 and later and pedigree from birth year 1950 on. Lactations shorter than 305 days are extended to 305 days. All records are preadjusted for effects of age at calving, month of calving, times milked per day, previous days open, and heterogeneous variance. Genetic evaluation is conducted using the single-trait BLUP repeatability model. The model includes fixed effects of management group (herd x year x season plus register status), parity x age, and inbreeding, and random effects of permanent environment and herd by sire interaction. PTAs are estimated and published four times a year (February, May, August, and November). PTAs are calculated relative to a five year stepwise base i.e., as a difference from the average of all cows born in the current year, minus five (5) years. Bull PTAs are published estimating daughter performance for bulls having at least 10 daughters with valid lactation records.

### Example 5

### Development of Economic Indexes

In total, 14 economic indexes were formed as the sum of the product of the trait economic weighting and trait PTA. For an individual (denoted as the j^{th} individual), its i^{th} index can be determined as follows:

$${I}_{ij} = {\sum }_{k = 1}^{12} {W}_{ik} * {PTA}_{kj} \qquad [ Equation 13 ]$$

where Wᵢₖ is the weight of k^{th} trait in the ith index (Tables 2 & 3), and PTAₖⱼ is the PTA of the k^{th} trait of the j^{th} individual.

TABLE 2
Economic weight of traditional traits in 14 economic indexes^{a,b,c}

| | Economic weight | | | | | | |
|---|---|---|---|---|---|---|---|
| Traits^{d} | index 1 | Index 2 | index 3 | Index 4 | index 5 | index 6 | index 7 |
| Milk yield | 0 | 0 | 0 | 0 | −0.01791 | −0.01202 | −0.02236 |
| Fat (lb) | 0.766667 | 0.5625 | −0.53082 | 0.46875 | 0.30625 | 0.46875 | 0.39375 |
| Fat (%) | 0 | 0 | 0 | 0 | 0 | 0 | 0 |
| Protein (lb) | 0.69697 | 0.8 | −0.9828 | 1.2 | 1.464 | 1.4 | 1.528 |
| Protein (%) | 0 | 0 | 0 | 0 | 0 | 0 | 0 |
| SCS | −45 | −25 | −1.69033 | −25 | 33.5 | 15 | 30.5 |
| PL | 8.09524 | 12 | −6.76247 | 6.66667 | 3.66667 | 3.33333 | 5.93333 |
| DPR | 6.42857 | 6.666667 | 12.82051 | 6.66667 | 4.4 | 5.33333 | 3.33333 |
| CA | 0.3 | 0 | 0 | 0 | 0 | 0 | 0 |
| Udder | 7.69231 | 0 | 0 | 0 | 0 | 0 | 0 |
| Feet/legs | 3.40909 | 0 | 0 | 0 | 0 | 0 | 0 |
| Body size | −4.25532 | 0 | 0 | 0 | 0 | 0 | 0 |

^{a}traits PTA is used to calculated economic weights
^{b}These economic weights were formed to appropriately measure values of different markets
^{c}unit used are same as the ones used in USDA AIPL genetic evaluation in February, 2007 (see www.aipl.arsusda.gov)
^{d}SCS = somatic cell score; PL = production lift; DPR = daughter pregnancy rate; CA = calving ability

TABLE 3
Economic weight of traditional traits in 14 economic indexes^{a,b,c}

| | Economic weight | | | | | | |
|---|---|---|---|---|---|---|---|
| Traits^{d} | index 8 | index 9 | index 10 | index 11 | index 12 | index 13 | index 14 |
| Milk yield | −0.01202 | −0.00841 | −0.00601 | −0.00901 | −0.00601 | 0.042067 | 0 |
| Fat (lb) | 0.46875 | 0.1875 | 0.1875 | 0.40625 | 0.46875 | 0 | 0.46875 |
| Fat (%) | 0 | 0 | 0 | 0 | 0 | 49.75124 | 0 |
| Protein (lb) | 1.4 | 0.72 | 0.8 | 1.52 | 1.2 | 0 | 1 |
| Protein (%) | 0 | 0 | 0 | 0 | 0 | 112.8668 | 0 |
| SCS | 15 | 70 | 50 | 60 | 40 | 50 | 50 |
| PL | 6.66667 | 4 | 5.33333 | 9.33333 | 10 | 0 | 3.33333 |
| DPR | 5.33333 | 8.66667 | 10 | 4.33333 | 6.66667 | 0 | 6.66667 |
| CA | 0 | 0 | 0 | 0 | 0 | 0 | 0 |
| Udder | 0 | 0 | 0 | 0 | 0 | 0 | 0 |
| Feet/legs | 0 | 0 | 0 | 0 | 0 | 0 | 0 |
| Body size | 0 | 0 | 0 | 0 | 0 | 0 | 0 |

^{a}traits PTA is used to calculated economic weights
^{b}These economic weights were formed to appropriately measure values of different markets
^{c}unit used are same as the ones used in USDA AIPL genetic evaluation in February, 2007 (see www.aipl.arsusda.gov)
^{d}SCS = somatic cell score; PL = production lift; DPR = daughter pregnancy rate; CA = calving ability

### Example 6

### Association Between Economic Indexes and SNPs

Selection of SNP loci: The SNP loci were selected by Affymetrix using proprietary algorithms designed to maximize the number, distribution and allele frequency of the loci. Of the 9919 SNPs represented in the Affymetrix chip, 9258 SNPs were derived from sequence data produced through the public bovine genome sequencing effort (Baylor College of Medicine) and 661 SNPs were derived from the IBISS (Interactive Bovine In Silico SNP) database (Hawken et al, 2004). An additional 22 SNPs were selected from the literature and represent 10 candidate genes associated with dairy traits.

Animal sample and genotyping. All Holstein bulls with a NAAB code were downloaded from USDA AIPL web site (www.aipl.arsusda.gov) and sent to several bull semen dealers to for semen purchase. A total of 3,145 Holstein bulls were selected from all purchased semen samples to form a resource population for this study. These samples represent multiple and overlapping sire and grandsire families.

Genotypic data for the 9919 Affymetrix SNPs was produced under contract by Affymetrix, Inc. using proprietary “Molecular Inversion Probe” (MIP) chemistry. Briefly, oligomers targeting each polymorphism are synthesized and hybridized to each genomic sample in a multiplex reaction. The discriminating SNP allele is added to the oligomer by gap-filling polymerization and ligation, followed by cleavage of the now circular oligomer. After amplification and labeling, the oligomers are hybridized to a microarray, scanned, and allele calls are determined. TaqMan® (Applied Biosystems, Foster City, Calif.) assays were designed by the manufacturer against the candidate genes SNPs, and were successful in delivering genotypes for 16 of the 22 polymorphisms. The remaining 6 SNPs (ABCG2, DGAT1, GH, PI-269, PI-989, and SPP1) were genotyped by Genaissance Pharmaceuticals (currently Clinical Data, Newton, Mass.) using Sequenom chemistry.

A total of 6967 SNPs from all SNPs genotyped as described above that were minimally sufficiently polymorphic were used in the analyses.

Trait phenotype & their preadjusments. The first steps were to download PTA data of traditional dairy traits of all progeny tested Holstein bulls from the USDA February 2007 genetic evaluation published at the AIPL site (www.aipl.arsusda.gov). The traditional dairy traits included milk yield (“MILK”; pounds), fat yield (“FAT”; pounds), fat percentage (“FATPCT”; percent), productive life (“PL”; months), somatic cell score (“SCS”; Log), daughter pregnancy rate (“DPR”; percent), protein yield (“PROT”; pounds), protein percentage (“PROTPCT”; percent), and net merit (“NM”; dollar). These PTA data were used to calculate the economic index for each bull using Equation 13. Please note that index1 is identical to NM.

Two types of analyses were performed: the first analysis used bulls' index value directly estimated from Equation 1 and is termed as analysis using unadjusted data; the first step of the second analysis was to adjust sire's effect, and is called analysis using preadjusted data.

The preadjustment of PTA was achieved using the following two models:

yᵢⱼ=sᵢ+Idᵢⱼ [Equation 14]

yᵢ=μ+β(SI)ᵢ+Idᵢ [Equation 15]

where yᵢ (yᵢⱼ) is the original index of the i^{th} bull (index of the j^{th} son of the i^{th} sire); sᵢ is the effect of the i^{th} sire; (SI), is the sire's index of the i^{th} bull of the whole sample; μ is the population mean; Idᵢ (Idᵢⱼ) is the residual bull index. For sires with <10 progeny tested sons, the sire effect was replaced with birth year effect.

Evaluation of associations between markers and economic indexes. Linkage Disequilibrium (LD) mapping was performed using analyses based on probabilities of individual ordered genotypes estimated conditional on observed marker genotypes. A stepwise procedure developed based on a likelihood ratio test was used for estimating probabilities of sire's ordered genotypes at all linked markers. The probabilities of ordered genotypes at loci of interest were estimated conditional on flanking informative markers as follows:

$$P ( {H}_{sik} {H}_{dlk} \vert M ) = {\sum }_{a} {\sum }_{b} P ( {H}_{sa} {H}_{db} \vert M ) * P ( {H}_{sik} {H}_{dlk} \vert {H}_{sa} {H}_{db} , M ) \qquad [ Equation 16 ]$$

where P(HₛₐH_{db}|M) is the probability of sire having a pair of haplotypes (or order genotype) HₛₐH_{db} at all linked loci conditional on the observed genotype data M, and P(HₛᵢₖH_{dlk}|HₛₐH_{db}, M) is the probability of a son having ordered genotype HₛᵢₖH_{dlk} at loci of interest conditional on sire's ordered genotype HₛₐH_{db} at all linked loci and the observed genotype data M.

To identify associations between haplotype probabilities and trait phenotypes, haplotypes of markers across each chromosome were defined by setting the maximum length of a chromosomal interval and minimum and maximum number of markers to be included. The association between pre-adjusted trait phenotypes and haplotype was evaluated via a regression approach with the following models:

$$\begin{matrix}{Id}_{k} = {\sum }_{i} {β}_{si} P ( {H}_{sik} ) + {e}_{k} & [ Equation 17 ] \\ {Id}_{k} = {\sum }_{i} {β}_{di} P ( {H}_{dik} ) + {e}_{k} & [ Equation 18 ] \\ {Id}_{k} = {\sum }_{i} {β}_{si} [ P ( {H}_{sik} ) + P ( {H}_{dik} ) ] + {e}_{k} & [ Equation 19 ] \\ {Id}_{k} = {\sum }_{i} {β}_{si} [ P ( {H}_{sik} {H}_{djk} ) + P ( {H}_{sjk} {H}_{dik} ) ] + {e}_{k} & [ Equation 20 ]\end{matrix}$$

where Idₖ is the index PTA and has different definition in different analyses: analysis using unadjusted data, Idₖ denotes the economic index calculated using Equation 13, In analysis using preadjusted data, Idₖ denotes preadjusted PTA of the k^{th} bull as defined in Equations 14 and 15 under the sire and SPTA model, respectively. eₖ is the residual; P(Hₛᵢₖ) and P(H_{dik}) are the probability of paternal and maternal haplotype of individual k being haplotype i; P(HₛᵢₖH_{dik}) is the probability of individual k having paternal haplotype i and maternal haplotype j that can be estimated using Equation 16; all β are corresponding regression coefficients. Equations 17, 18, 19, and 20 are designed to model paternal haplotype, maternal haplotype, additive haplotype, and genotype effects, respectively.

The analyses were performed for each SNP and all combination of two SNPs that are from the same chromosome and the distance between them is estimated to be ≦2 cM. It should be noted that haplotype probabilities in Equations 17 to 20 become allele probabilities in cases of single marker analyses.

Least-squares methods were used to estimate the effect of a haplotype or haplotype pair on a phenotypic trait and the regular F-test used to test the significance of the effect. Permutation tests were performed based on phenotype permutation (20,000) within each paternal half-sib family to estimate Type I error rate (p value)

### Example 7

### Identification of Markers Valuable for Predicting Economic Indexes

The first step was to mine the results obtained from analyses using pre-adjusted data, both sire and SPTA model, to pick SNPs valuable in predicting economic indexes, which was designed for more robust results. SNP selection was based on multiple factors, including: allelic frequency of each SNP; statistical evidence for an association between a SNP of interest and an economic index; and for an association between a 2 SNP locus group that contains the SNP of interest and an economic index, the statistical evidence for association with all 14 economic indexes as described above; and a joint consideration of all SNPs within 10 to 20 cM for each SNP choice in the genome.

The economic weights in the genomic marker index for genotypes at each SNP selected were then determined based on results from using unadjusted data, which was designed to achieve higher accuracy of genetic merit prediction. Specifically, weights were estimated as the allelic effect in single marker analyses as described in Equation 19.

In total, 121 markers were identified in our first analysis, and the estimated allelic economic weights of these 121 markers for all 14 economic indexes are reported in Tables 2 and 3. Please note that the weight of a genotype is calculated as the sum of the weights of two alleles of which the genotype consists.

### Example 8

### Determination of a Genomic Marker Index of a Bull

Equation 2 is used to calculate the genomic marker index, in conjunction with Table 1. Specifically, the variables in the equation are defined by the weighted coefficients listed in the table for each respective marker.

The first step is to genotype all of 121 markers that are described in Table 1 for an animal. With the resulting genotype data, the i^{th} genomic marker index of the animal (i.e., the k^{th} animal) can be determined using following equation:

$${GMI}_{ik} = {\sum }_{j = 1}^{121} {W}_{ij} ( {G}_{jk} ) \qquad [ Equation 2 ]$$

where Gⱼₖ is the genotype of j^{th} marker of bull k; Wᵢⱼ(Gⱼₖ) is the weight of genotype Gⱼₖ at the j^{th} marker for index i. The values listed in table 1 correspond to the weighting for a single strand of DNA. Therefore, each genotype will have two values for each SNP, one for each allele. A homozygous value will be two times the weighting for the respective allele, while a heterozygous value will be the sum of each allele weighting. For example, a sample which is homozygous for the G allele at SNP1 (e.g., GG) would include a weighting equal to 2× the weighting listed for the G allele in table 1. A sample which is heterozygous for the SNP1 (e.g., GA) would include a weighting equal to the sum of the weighting for the G allele and the weighting for the A allele.

For example, the GMI for index 1 of a bull would be calculated as follows:

Genotype of SNP 1=GG, weighting=0.45621+0.45621=0.91242

Genotype of SNP 2=GA, weighting=0.174516+0.480119=0.657895

Genotype of SNP 3=TT, weighting=(−0.13095)+(−0.13095)=−0.26191
. . .

Genotype of SNP 121=AG, weighting=0.642706+0.071233=0.713936

Therefore, GMI₁=0.91242+0.657895+(−0.26191)+0.713936

### Example 9

### Determination of a Genomic Marker Index of Bull Semen

Even though semen contains haploid cells, they can still be used with the GMI by genotyping a large number of cells. The first step is to get a semen straw or sample that contains sufficiently large number of sperm cells (e.g., >1,000,000 cells). The second step is to extract DNA from the semen straw (namely a pool of a large number of sperm cells). The extracted DNA is then to be used to genotype markers listed in Table 1 and the Sequence Listing. These genotype results will include information on both strands of DNA of the parent animal. Therefore, the genotype data can be used for Genomic marker index calculation using Equation 3.

### REFERENCES

The references cited in this application, both above and below, are specifically incorporated herein by reference.

### Non-Patent Literature

Abdel-Azim, G and Freeman, A E, (2002) J. Dairy Sci. 85:1869-1880.
Blott, S., Kim, J. J., Moisio, S., et al. (2003). Genetics 163: 253-266.
Ciobanu, D C, Bastiaansen, J W M, Longergan, S M, Thomsen, H, Dekkers, J C M, Plastow, G S, and Rothschild, M F, (2004) J. Anim. Sci. 82:2829-39.
Cohen-Zinder, M. et al. (2005) Genome Res. 15:936-44.
Davis, GP and DeNise, S K, (1998) J. Anim. Sci. 76:2331-39.
Dekkers, J C M, and Chakraborty, R, (2001) J. Anim. Sci. 79:2975-90.
Demars J, Riquet J, Feve K, Gautier M, Morisson M, Demeure O, Renard C, Chardon P, Milan D. (2006), BMC Genomics, 24:7-13
Du and Hoeschele, (2000) Genetics 156:2051-62.
Ducrocq, V. 1987. An analysis of length of productive life in dairy cattle. Ph.D. Diss. Cornell Univ., Ithaca, N.Y.; Univ. Microfilms Int., Ann Arbor, Mich.
Everts-van der Wind A, Larkin D M, Green C A, Elliott J S, Olmstead C A, Chiu R, Schein J E, Marra M A, Womack J E, Lewin H A. (2005) Proc Natl Acad Sci USA, Dec 20 102(51):18526-31.
Falconer, D S, and Mackay, T F C, (1996) Introduction to Quantitative Genetics. Harlow, UK: Longman.
Fernando, R, and Grossman, M, (1989) Marker assisted selection using best linear unbiased prediction. Genetics Selection Evolution 21:467-77.
Franco, M M, Antunes, R C, Silva, H D, and Goulart, L R (2005) J. Appl. Genet. 46(2):195-200.
Grisart, B. et al. (2002) Genome Res. 12:222-231
Grosz, M D, Womack, J E, and Skow, L C (1992) Genomics, 14(4):863-868.
Hayes, B, and Goddard, M E, (2001) Genet. Sel. Evol. 33:209-229.
Hayes, B, Chamberlain, A. J., Goddard, M. E. (2006) Proc. 8^{th} WCGALP 22:(16).
Kaminski, S, Ahman, A, Ruse, A, Wojcik, E, and Malewski, T (2005) J. Appl. Genet. 46(1):45-58.
Kuhn, C. et al. (2004). Genetics 167:1873-81.
Kwok P Y, Methods for genotyping single nucleotide polymorphisms, (2001), Annu. Rev. Genomics Hum. Genet., 2:235-258.
Meuwissen, T H E, and Van Arendonk, J A M, (1992) J Dairy Sci. 75:1651-1659.
Meuwissen, T H E, Hayes, B J, and Goddard, M E, (2001) Genetics. 157:1819-29.
Rothschild and Plastow, (1999), AgBioTechNet 10:1-8.
Schaeffer, L R (2006) J. Anim. Breed. Genet. 123:218-223.
Schnabel, R. et al. (2005) PNAS 102:6896-6901.
Schrooten, C, Bovenhuis, H, van Arendonk, J A M, and Bijma, P (2005) J. Dairy Sci. 88:1569-1581.
Sharma, B S, Jansen, G B, Karrow, N A, Kelton, D, and Jiang, Z, (2006) J. Dairy Sci. 89:3653-3663.
Short, T H, et al. (1997) J. Anim. Sci. 75:3138-3142.
Spelman, R J and Bovenhuis, H, (1998) Animal Genetics, 29:77-84.
Spelman, R J and Garrick, D J, (1998) J Dairy Sci, 81:2942-2950.
Stearns, T M, Beever, J E, Southey, B R, Ellis, M, McKeith, F K and Rodriguez-Zas, S L, (2005) J. Anim. Sci. 83:1481-93.
Syvänen A C, Accessing genetic variation: genotyping single nucleotide polymorphisms, (2001) Nat. Rev. Genet. 2:930-942.
VanRaden, P. M. and E. J. H. Klaaskate. 1993. J. Dairy Sci. 76:2758-2764.
Verrier, E, (2001) Genet. Sel. Eva 33:17-38.
Villanueva, B, Pong-Wong, R, Fernandez, J, and Toro, M A (2005) J. Anim. Sci. 83:1747-52.
Weller J I, Kashi Y, Soller M. (1990) J. Dairy Sci. 73:2525-37
Williams, J L, (2005), Rev. Sci. Tech. Off. Int. Epiz. 24(1):379-391.
Windig, J J, and Meuwissen, T H E, (2004) J. Anim. Breed. Genet. 121:26-39.
Winter, A. et al. (2002). PNAS, 99:9300-9305.
Womack, J, (1987), Dev. Genet. 8(4):281-293.
Yasue H, Kiuchi S, Hiraiwa H, Ozawa A, Hayashi T, (2006), Cytogenet. Genome Res., 112(1-2):121-125.
Youngerman, S M, Saxton, A M, Oliver, S P, and Pighetti, G M, (2004) J. Dairy Sci. 87:2442-2448.
Zhang, K, Martiny, A C, Reppas, N B, Barry, K W, Malek, J, Chisholm, S W, Church, G M “Sequencing genomes from single cells by polymerase cloning” Nature Biotechnology 24, 680-686 (2006)
Zondervan, K and Cardon, L (2004) Nat Rev Genet. 2004 February; 5(2):89-100

Patent Literature

| Publication/ | | | |
|---|---|---|---|
| Patent Number | Title | Inventors | Pub. Date |
| U.S. Pat. No. 5,041,371 | Genetic marker for | Cowan, Charles M.; | Aug. 20, 1991 |
| | superior milk products in | Dentine, Margaret R.; | |
| | dairy cattle | Ax, Roy L.; Schuler, | |
| | | Linda A. | |
| U.S. Pat. No. 5,374,523 | Allelic variants of bovine | Collier, Robert J.; | Dec. 20, 1994 |
| | somatotropin gene:genetic | Hauser, Scott D.; Krivi, | |
| | marker for superior milk | Gwen G.; Lucy, | |
| | production in bovine | Matthew C. | |
| U.S. Pat. No. 5,582,987 | Methods for testing bovine | Lewin, Harris A.; van | Dec. 10, 1996 |
| | for resistance or | Eijk, Michiel J. T. | |
| | susceptibility to persistent | | |
| | lymphocytosis by detecting | | |
| | polymorphism in BoLA- | | |
| | DR3 exon 2 | | |
| U.S. Pat. No. 5,614,364 | Genetic marker for | Tuggle, Christopher K.; | Mar. 25, 1997 |
| | improved milk production | Freeman, Albert E. | |
| | traits in cattle | | |
| US2003039737A1 | Population of dairy cows | Cooper, Garth J. S. | Feb. 27, 2003 |
| | producing milk with | | |
| | desirable characteristics | | |
| | and methods of making | | |
| | and using same | | |
| US2004076977A1 | Marker assisted selection | Georges, Michel | Apr. 22, 2004 |
| | of bovine for improved | Alphonse Julien; | |
| | milk production using | Coppieters, Wonter | |
| | diacylglycerol | Herman Robert; Grisart, | |
| | acyltransferase gene dgat1 | Bernard Marie-Josee Jean; | |
| | | Shell, Russell Grant; Jean | |
| | | Reid, Suzanne; Ford, | |
| | | Christine Ann; Spelman, | |
| | | Richard John | |
| US2004091933A1 | Methods for genetic | Stoughton, Roland; | May 13, 2004 |
| | interpretation and | Marton, Matthew J. | |
| | prediction of phenotype | | |
| US2004112299A1 | Incorporation of | Muir, William M | Jun. 17, 2004 |
| | competitive effects in | | |
| | breeding program to | | |
| | increase performance | | |
| | levels and improve animal | | |
| | well being | | |
| US2004234986A1 | Method of testing a | Fries, Hans-Rudolf; | Nov. 25, 2004 |
| | mammal for its | Winter, Andreas | |
| | predisposition for fat | | |
| | content of milk and/or its | | |
| | predisposition for meat | | |
| | marbling | | |
| US2004235061A1 | Methods for selecting and | Wilkie, Bruce N.; | Nov. 25, 2004 |
| | producing animals having a | Mallard, Bonnie A | |
| | predicted level of immune | | |
| | response, disease resistance | | |
| | or susceptibility, and/or | | |
| | productivity | | |
| US2004241723A1 | Systems and methods for | Marquess, Foley Leigh | Dec. 2, 2004 |
| | improving protein and milk | Shaw; Laarveld, Bernard; | |
| | production of dairy herds | Cleverly Buchanan, | |
| | | Fiona; Van Kessel, | |
| | | Andrew Gerald; Schmutz, | |
| | | Sheila Marie; Waldner, | |
| | | Cheryl; Christensen, | |
| | | David | |
| US2004254104A1 | Marker assisted selection | Blott, Sarah; Kim, Jong- | Dec. 16, 2004 |
| | of bovine for improved | Joo; Schmidt-Kuntzel, | |
| | milk composition | Anne; Cornet, Anne; | |
| | | Berzi, Paulette; | |
| | | Cambisano, Nadine; | |
| | | Grisart, Bernard; Karim, | |
| | | Latifa; Simon, Patricia; | |
| | | Georges, Michel; Farnir, | |
| | | Frederic; Coppieters, | |
| | | Wouter; Moisio, Sirja; | |
| | | Vilkki, Johanna; | |
| | | Spelman, Richard; | |
| | | Johnson, Dave; Ford, | |
| | | Christine; Snell, Russell | |
| US2005123929A1 | Methods and compositions | Khatib, Hasan | Jun. 9, 2005 |
| | for genetically detecting | | |
| | improved milk production | | |
| | traits in cattle | | |
| US2005136440A1 | Method for identifying | Renaville, Robert; | Jun. 23, 2005 |
| | animals for milk | Gengler, Nicolas | |
| | production qualities by | | |
| | analysing the | | |
| | polymorphism of the Pit-1 | | |
| | and kappa-casein genes | | |
| US2005137805A1 | Gene expression profiles | Lewin, Harris A.; Liu, | Jun. 23, 2005 |
| | that identify genetically | Zonglin; Rodriguez-Zas, | |
| | elite ungulate mammals | Sandra; Everts, Robin E. | |
| US2005153317A1 | Methods and systems for | DeNise, Sue; Rosenfeld, | Jul. 14, 2005 |
| | inferring traits to breed and | David; Kerr, Richard; | |
| | manage non-beef livestock | Bates, Stephen; Holm, | |
| | | Tom | |
| US2006037090A1 | Selecting animals for | Andersson, Leif; | Feb. 16, 2006 |
| | desired genotypic or | Andersson, Goran; | |
| | potential phenotypic | Georges, Michel; Buys, | |
| | properties | Nadine | |
| US2006094011A1 | Method for altering fatty | Morris, Christopher | May 4, 2006 |
| | acid composition of milk | Anthony; Tate, Michael | |
| | | Lewis | |
| US2006121472A1 | Method for determining the | Prinzenberg, Eva-Maria; | Jun. 8, 2006 |
| | allelic state of the 5′-end of | Erhardt, George | |
| | the $g(a)s1-casein gene | | |
| US2006166244A1 | DNA markers for increased | Schnabel, Robert D.; | Jul. 27, 2006 |
| | milk production in cattle | Sonstegard, Tad S.; Van | |
| | | Tassell, Curtis P.; | |
| | | Ashwell, Melissa S.; | |
| | | Taylor, Jeremy F. | |
| US2007105107A1 | Marker assisted best linear | Wang, Tianlin; Lohuis, | May 10, 2007 |
| | unbiased prediction (mablup): | Michael M.; Kojima, | |
| | software adaptions | Cheryl J.; Du, Fengxing; | |
| | for large breeding | Byatt, John C. | |
| | populations in farm animal | | |
| | species | | |
| WO0202822A2 | METHOD OF | TAYLOR, Jeremy, | Jan. 10, 2002 |
| | MANAGING AND | Francis; DAVIS, Scott, K.; | |
| | MARKETING | DAVIS, Sara, L., F.; | |
| | LIVESTOCK BASED ON | LIND, Luke | |
| | GENETIC PROFILES | | |
| WO0236824A1 | MARKER ASSISTED | GEORGES, MICHEL, | May 10, 2002 |
| | SELECTION OF BOVINE | ALPHONSE, JULIEN; | |
| | FOR IMPROVED MILK | COPPIETERS, | |
| | PRODUCTION USING | WOUTER, HERMAN, | |
| | DIACYLGLYCEROL | ROBERT; GRISART, | |
| | ACYLTRANSFERASE | BERNARD, MARIE- | |
| | GENE DGAT1 | JOSEE, JEAN; SNELL, | |
| | | RUSSELL, GRANT; | |
| | | REID, SUZANNE, JEAN; | |
| | | FORD, CHRISTINE, | |
| | | ANN; SPELMAN, | |
| | | RICHARD, JOHN | |
| WO03104492A1 | MARKER ASSISTED | BLOTT, SARAH; KIM, | Dec. 18, 2003 |
| | SELECTION OF BOVINE | JONG-JOO; SCHMIDT- | |
| | FOR IMPROVED MILK | KUNTZEL, ANNE; | |
| | COMPOSITION | CORNET, ANNE; | |
| | | BERZI, PAULETTE; | |
| | | CAMBISANO, NADINE; | |
| | | GRISART, BERNARD; | |
| | | KARIM, LATIFA; | |
| | | SIMON, PATRICIA; | |
| | | GEORGES, MICHEL; | |
| | | FARNIR, FREDERIC; | |
| | | COPPIETERS, | |
| | | WOUTER; MOISIO, | |
| | | SIRJA; VILKKI, | |
| | | JOHANNA; JOHNSON, | |
| | | DAVE; SPELMAN, | |
| | | RICHARD; FORD, | |
| | | CHRISTINE; SNELL, | |
| | | RUSSELL | |
| WO04004450A1 | METHOD FOR | MORRIS, Christopher | Jan. 15, 2004 |
| | ALTERING FATTY | Anthony; TATE, Michael | |
| | ACID COMPOSITION OF | Lewis | |
| | MILK | | |
| WO04048609A2 | METHODS AND KITS | RENAVILLE, Robert; | Jun. 10, 2004 |
| | FOR THE SELECTION | PARMENTIER, Isabelle | |
| | OF ANIMALS HAVING | | |
| | CERTAIN MILD | | |
| | PRODUCTION | | |
| | CAPABILITIES, BASED | | |
| | ON THE ANALYSIS OF | | |
| | A POLYMORPHISM IN | | |
| | THE SOMATOTROPIN | | |
| | RECEPTOR GENE | | |
| WO04083456A1 | SYSTEMS AND | MARQUESS, Foley, | Sep. 30, 2004 |
| | METHODS FOR | Leigh, Shaw; | |
| | IMPROVING PROTEIN | LAARVELD, Bernard; | |
| | AND MILK | CLEVERLY | |
| | PRODUCTION OF | BUCHANAN, Fiona; | |
| | DAIRY HERDS | VAN KESSEL, Andrew, | |
| | | Gerald; SCHMUTZ, | |
| | | Sheila, Marie; | |
| | | WALDNER, Cheryl; | |
| | | CHRISTENSEN, David | |
| WO05007881A2 | IMPROVING | SCHMUTZ, SHEILA | Jan. 27, 2005 |
| | PRODUCTION | MARIE; GOODALL, | |
| | CHARACTERISTICS OF | JULIE JANINE | |
| | CATTLE | | |
| WO05030789A1 | ADRENERGIC | COLLIER, Robert, J.; | Apr. 7, 2005 |
| | RECEPTOR SNP FOR | LOHIUS, Michael; | |
| | IMPROVED MILKING | GROSZ, Michael | |
| | CHARACTERISTICS | | |
| WO05040400A2 | METHODS AND | DENISE, Sue, K.; | May 6, 2005 |
| | SYSTEMS FOR | ROSENFELD, David; | |
| | INFERRING TRAITS TO | KERR, Richard; BATES, | |
| | MANAGE NON-BEEF | Stephen; HOLM, Tom | |
| | LIVESTOCK | | |
| WO05056758A2 | METHODS AND | KHATIB, Hasan | Jun. 23, 2005 |
| | COMPOSITIONS FOR | | |
| | GENETICALLY | | |
| | DETECTING IMPROVED | | |
| | MILK PRODUCTION | | |
| | TRAITS IN CATTLE | | |
| WO05089122A2 | ANIMALS WITH | JOHNSON, Geoffrey, B.; | Sep. 29, 2005 |
| | REDUCED BODY FAT | PLATT, Jeffrey, L.; | |
| | AND INCREASED BONE | JOHNSON, Joel, W. | |
| | DENSITY | | |
| WO06076419A1 | DNA MARKERS FOR | TAYLOR, Jeremy, F.; | Jul. 20, 2006 |
| | CATTLE GROWTH | SCHNABEL, Robert, D. | |
| WO06076563A2 | DNA MARKERS FOR | SCHNABEL, Robert, D.; | Jul. 20, 2006 |
| | INCREASED MILK | SONSTEGARD, Tad, S.; | |
| | PRODUCTION IN | VAN TASSELL, Curtis, P.; | |
| | CATTLE | ASHWELL, Melissa, S.; | |
| | | TAYLOR, Jeremy, F. | |
| WO9213102A1 | POLYMORPHIC DNA | Georges, Michel; | Aug. 6, 1992 |
| | MARKERS IN BOVIDAE | MASSEY, Joseph, M. | |
| WO9319204A1 | BOVINE ALLELES AND | LEWIN, Harris, A.; VAN | Sep. 30, 1993 |
| | GENETIC MARKERS | EIJK, Michiel, J., T. | |
| | AND METHODS OF | | |
| | TESTING OF AND | | |
| | USING SAME | | |
| WO9403641A1 | GENETIC MARKER FOR | COLLIER, Robert, | Feb. 17, 1994 |
| | DAIRY CATTLE | Joseph; HAUSER, Scott, | |
| | PRODUCTION | David; KRIVI, Gwen, | |
| | SUPERIORITY | Grabowski; LUCY, | |
| | | Matthew, Christian | |
| WO9414064A1 | METHODOLOGY FOR | KENNEDY, Brian, | Jun. 23, 1994 |
| | DEVELOPING A | Wayne; WILKIE, Bruce, | |
| | SUPERIOR LINE OF | Nicholson; MALLARD, | |
| | DOMESTICATED | Bonnie, Allorene | |
| | ANIMALS | | |
| WO27064935A2 | ONLINE | HODNETT, Michael; | Jun. 7, 2007 |
| | MARKETPLACE FOR | HAWTHORNE, Louis | |
| | ANIMAL GENETICS | | |

1. A method for allocating a bovine animal for use according to the animal's genomic marker index, the method comprising:
a. determining the animal's genotype at ten or more locus/loci; wherein at least one locus contains a single nucleotide polymorphism (SNP), having at least two allelic variants; and wherein at least 10 SNPs are: selected from the SNPs described in Table 1 and the Sequence Listing;
b. analyzing the determined genotype of at least one evaluated animal at 10 or more SNPs selected from the SNPs described in Table 1 and the Sequence Listing; and/or
c. calculating a genomic marker index using Equation 2 and the weightings found in Table 1:
$${GMI}_{ik} = {\sum }_{j = 1}^{121} {W}_{ij} ( {G}_{jk} ) \qquad [ Equation 2 ]$$
wherein Gⱼₖ is the genotype of j^{th} marker of animal k and Wᵢⱼ(Gⱼₖ) is the weight of genotype Gⱼₖ at the j^{th} marker for index i.
d. allocating the animal for use based on its determined genomic marker index.

2. The method of claim 1 wherein the animal's genotype is evaluated at 20 or more loci that contain SNPs selected from the SNPs described in Table 1 and the Sequence Listing;

3. The method of claim 2 wherein the animal's genotype is evaluated at 50 or more loci.

4. The method of claim 2 wherein the animal's genotype is evaluated at 100 or more loci.

5. The method of claim 2 wherein the animal's genotype is evaluated at 200 or more loci.

6. The method of 2 wherein the animal's genotype is evaluated at 500 or more loci.

7. The method of claim 1 that comprises whole-genome analysis

8. The method of claim 1 further comprising analysis using pedigree information, progeny information, breed information, or phenotypic information prior to allocation.

9. The method of any of claims 1 through 8 that further comprise isolating sperm from said animal.

10. The method of claim 9 further comprising altering a sex-determining characteristic of said isolated sperm.

11. The method of claim 9 further comprising freezing said isolated sperm.

12. A method for selecting a potential parent bovine animal based on a genomic marker index, comprising:
a. determining at least one potential parent animal's genotype at 10 or more genomic loci; wherein at least one locus contains a single nucleotide polymorphism (SNP) that has at least two allelic variants, and wherein at least ten SNPs are selected from the SNPs described in Table 1 and the Sequence Listing;
b. analyzing the determined genotype of at least one evaluated animal for ten or more SNPs selected from the SNPs described in Table 1;
c. calculating a genomic marker index using weightings found in Table 1, and the following equation:
$${GMI}_{ik} = {\sum }_{j = 1}^{121} {W}_{ij} ( {G}_{jk} ) \qquad [ Equation 2 ]$$
wherein Gⱼₖ is the genotype of j^{th} marker of animal k and Wᵢⱼ(Gⱼₖ) is the weight of genotype Gⱼₖ at the j^{th} marker for index i.
d. allocating at least one animal for breeding use based on its genotype.

13. The method of claim 12 wherein the potential parent animal's genotype is evaluated at 20 or more loci that contain SNPs selected from the SNPs described in Table 1

14. The method of claim 12 wherein the potential parent animal's genotype is evaluated at 50 or more loci.

15. The method of claim 12 wherein the potential parent animal's genotype is evaluated at 100 or more loci.

16. The method of claim 12 wherein the potential parent animal's genotype is evaluated at 200 or more loci.

17. The method of claim 12 wherein the potential parent animal's genotype is evaluated at 500 or more loci.

18. The method of claim 12 that further comprise whole-genome analysis.

19. The method of claim 12 that further comprise isolating sperm from said animal.

20. The method of claim 19 further comprising altering a sex-determining characteristic of said isolated sperm.

21. The method of claim 19 further comprising freezing said isolated sperm.

22-56. (canceled)
